(19) United States
(12) Patent Application Publication
(10) Pub. No.: US 2026/0207547 A1
(43) Pub. Date: Jul. 23, 2026

(54) USE OF UGONIN COMPOUND FOR TREATING JOINT-RELATED DISEASES

(71) Applicants: National Sun Yat-sen University, Kaohsiung (TW); China Medical University, Taichung (TW)

(72) Inventors: Chih-Chuang LIAW, Kaohsiung (TW); Chih-Hsin Tang, Taichung (TW)

(21) Appl. No.: 19/450,874

(22) Filed: Jan. 16, 2026

Related U.S. Application Data
(60) Provisional application No. 63/747,370, filed on Jan. 21, 2025.

(51) Int. Cl. A61K 31/352; A61P 19/02

(52) U.S. Cl. CPC A61K 31/352; A61P 19/02

(57) ABSTRACT

The present invention provides a use of a ugonin compound for the treatment of joint-related diseases by enhancing chondrogenesis. More specifically, the chondrogenesis is mediated by the ugonin compound’s ability to promote the increase of aggrecan and type II collagen, which is achieved through the inhibition of miR-3074-5p and the activation of the MAPK signaling pathway.

### CROSS-REFERENCE TO RELATED APPLICATION

This application claims the benefit of U.S. Provisional Application Ser. No. 63/747, 370, filed January 21, 2025. The disclosure of the above application is incorporated herein by reference in its entirety.

### FIELD

The present invention relates to natural products or synthetic compounds and pharmaceutical uses thereof; more particularly, it relates to a use of a ugonin compound in the treatment of arthritis.

### BACKGROUND

As one of the essential components of the human musculoskeletal system, joints are responsible for connecting bones and providing a corresponding range of motion, thereby enabling the human body to perform various movements such as walking, running, jumping, and bending. Meanwhile, in addition to being the activity hubs of the human body, joints are also the primary load-bearing sites. Especially during intense exercise, joints are typically subjected to multiple pressures from both body weight and external forces.

There are numerous joint-related diseases, among which arthritis is particularly problematic in modern times. The primary characteristics of arthritis include joint inflammation and pain, and it is categorized into types such as osteoarthritis, rheumatoid arthritis, and gouty arthritis. Among these, osteoarthritis is the most common type, accounting for over 50% of all arthritis patients, with mechanical wear of the joints being the primary cause. According to statistics from the Ministry of Health and Welfare (Taiwan), the prevalence rate among individuals over 70 years of age reaches over 70%. Rheumatoid arthritis is an autoimmune disease affecting approximately 0.5% to 1% of the global adult population, with a higher incidence in females. Gouty arthritis is closely associated with metabolic issues, with a global prevalence ranging from approximately 1% to 3%, occurring more frequently in males, particularly those with hyperuricemia. Given that joints are essential for human activity and a significant proportion of the population suffers from joint-related diseases, treatment and recovery methods for joints are currently in urgent demand.

### SUMMARY

One concept of the present invention is to resolve the aforementioned problems by utilizing a ugonin compound, which is derived from Helminthostachys zeylanica. Notably, the use of such compounds in the treatment of joint-related diseases has not been previously addressed.

The present invention provides a method for preventing or treating a joint-related disease, comprising administering to a subject in need a composition comprising a ugonin compound, wherein the ugonin compound enhances chondrogenesis. In some embodiments, the ugonin compound is selected from the group consisting of ugonin J, ugonin K, ugonin L, ugonin M, ugonin N, ugonin O, ugonin P, ugonin S, ugonin T, ugonin U, and ugonin V.

In some embodiments, the joint-related disease is selected from the group consisting of arthritis, chondrodysplasia, chondral injury, chondromalacia patella, chondritis, and chondrolysis. In some embodiments, the arthritis is selected from the group consisting of osteoarthritis, rheumatoid arthritis, metabolic arthritis, psoriatic arthritis, and ankylosing spondylitis.

In some embodiments, the ugonin compound promotes a cartilage-forming cell to generate an extracellular matrix to enhance chondrogenesis. In some embodiments, the cartilage-forming cell includes chondroblast, chondrocyte, or any combination thereof. In some embodiments, the extracellular matrix includes collagen, glycosaminoglycan, and proteoglycan. Specifically, in some embodiments, the collagen includes type II collagen, and the proteoglycan includes aggrecan.

In some embodiments, the ugonin compound suppresses expression of a microRNA in a cartilage-forming cell to enhance chondrogenesis, wherein the microRNA includes miR-3074-5p.

In some embodiments, the method further comprises: administering the composition via an administration route to the subject in need to reach an effective concentration of the ugonin compound in a biofluid thereof. In some embodiments, the administration route comprises intra-articular injection. In some embodiments, the effective concentration ranges from 0.1μM to 100μM. In some embodiments, the biofluid comprises synovial fluid.

The present invention further provides a composition for preventing or treating a joint-related disease, the composition comprising a ugonin compound selected from the group consisting of ugonin J, ugonin K, ugonin L, ugonin M, ugonin N, ugonin O, ugonin P, ugonin S, ugonin T, ugonin U, and ugonin V. In some embodiments, the joint-related disease is selected from the group consisting of arthritis, chondrodysplasia, chondral injury, chondromalacia patella, chondritis, and chondrolysis. In some embodiments, the ugonin compound is ugonin P. In some embodiments, the composition is in a dosage form formulated for intra-articular injection.

The advantageous effects of the present invention include: the ugonin compound and the composition comprising the same can be used to treat the aforementioned joint-related diseases by a mechanism that includes promoting chondrogenesis. More specifically, the enhancement of chondrogenesis results from the ability of the ugonin compound to increase aggrecan and type II collagen. Furthermore, the ugonin compound achieves these effects by suppressing miR-3074-5p and activating the MAPK signaling pathway. It should be understood that the effects of the present invention are not limited thereto.

### BRIEF DESCRIPTION OF THE DRAWINGS

In some drawings, healthy subjects are represented as "NL", osteoarthritis patients are represented as "OA", and rheumatoid arthritis patients are represented as "RA".

FIG. 1A to FIG. 1J represent chemical structures of ugonin J, ugonin K, ugonin L, ugonin M, ugonin N, ugonin O, ugonin P, ugonin S, ugonin T, ugonin U, and ugonin V, respectively.

FIG. 2A is a heatmap showing the expression differences of genes related to extracellular matrix (ECM) and cartilage degradation among healthy subjects, OA patients, and RA patients in the GSE55235 dataset.

FIG. 2B is a KEGG enrichment analysis plot showing differences in signaling pathways, where arrows point to, from top to bottom: degradation of the extracellular matrix, extracellular matrix organization, collagen degradation, and activation of matrix metalloproteinases.

FIG. 2C is an IPA pathway analysis enrichment plot showing differences in signaling pathways, where the dashed box includes, from top to bottom: collagen degradation, inhibition of matrix metalloproteinases, and degradation of the extracellular matrix.

FIG. 2D shows the gene values of aggrecan in healthy subjects, OA patients, and RA patients, where the values are obtained from the GSE55235 dataset of the GEO database, with statistical significance *p < 0.05.

FIG. 2E shows the gene values of type II collagen in healthy subjects, OA patients, and RA patients, where the values are obtained from the GSE55235 dataset of the GEO database, with statistical significance *p < 0.05.

FIG. 2F is a distribution plot (e.g., t-SNE or UMAP) where different colored blocks represent different chondrocyte types in human cartilage tissue, obtained from the GSE55460 dataset.

FIG. 2G is a distribution plot showing the gene expression level of aggrecan in different chondrocyte types in human cartilage tissue, where the human cartilage tissue refers to healthy subjects and OA patients.

FIG. 2H is a distribution plot showing the gene expression level of type II collagen in different chondrocyte types in human cartilage tissue, where the human cartilage tissue refers to healthy subjects and OA patients, and the differences are obtained by comparing single-cell gene expression data.

FIG. 3A shows the cell viability of ATDC5 cells treated with different concentrations of ugonin P, where the 0 μM group is the control group (ratio of 100%) for relative comparison with other experimental groups (0.3μM, 1μM, 3μM).

FIG. 3B shows the cell viability of primary human chondrocytes treated with different concentrations of ugonin P, where the 0 μM group is the control group (ratio of 100%) for relative comparison with other experimental groups (0.3μM, 1μM, 3μM).

FIG. 3C shows the mRNA expression levels of aggrecan and type II collagen relative to the control group in ATDC5 cells after treatment with different concentrations of ugonin P. In FIG. 3C, the left bar for each concentration represents aggrecan, and the right bar represents type II collagen.

FIG. 3D shows the mRNA expression levels of aggrecan and type II collagen relative to the control group in primary human chondrocytes after treatment with different concentrations of ugonin P. In FIG. 3D, the left bar for each concentration represents aggrecan, and the right bar represents type II collagen.

FIG. 3E is a representative Western blot image showing the protein expression levels of aggrecan and type II collagen in ATDC5 cells after treatment with different concentrations of ugonin P, with β-actin serving as an internal control.

FIG. 3F is a bar graph showing the differences in protein expression levels of aggrecan and type II collagen in ATDC5 cells after treatment with different concentrations of ugonin P. In FIG. 3F, the left bar for each concentration represents aggrecan, and the right bar represents type II collagen.

FIG. 3G is a representative Western blot image showing the protein expression levels of aggrecan and type II collagen in primary human chondrocytes after treatment with different concentrations of ugonin P, with β-actin serving as an internal control.

FIG. 3H is a bar graph showing the differences in protein expression levels of aggrecan and type II collagen in primary human chondrocytes after treatment with different concentrations of ugonin P. In FIG. 3H, the left bar for each concentration represents aggrecan, and the right bar represents type II collagen.

FIG. 3I illustrates the mRNA expression of aggrecan in ATDC5 cells treated with different doses of ugonin P.

FIG. 3J illustrates the mRNA expression of type II collagen in ATDC5 cells treated with different doses of ugonin P.

FIG. 3K shows Alcian blue staining results illustrating the dose-dependent relationship between ugonin P and Alcian blue substrates in ATDC5 cells, with a treatment time of 2 weeks.

FIG. 3L is a quantitative bar graph of FIG. 3K, presented as absorbance values at a wavelength of 620nm.

FIG. 4A shows the intersection of miRNAs that can simultaneously bind to aggrecan and COL2.

FIG. 4B provides the expression levels of miR-18a-3p in different groups obtained from the GEO database.

FIG. 4C provides the expression levels of miR-497-5p in different groups obtained from the GEO database.

FIG. 4D provides the expression levels of miR-3074-5p in different groups obtained from the GEO database.

FIG. 4E provides the expression levels of miR-18a-3p in different groups detected by qPCR.

FIG. 4F provides the expression levels of miR-497-5p in different groups detected by qPCR.

FIG. 4G provides the expression levels of miR-3074-5p in different groups detected by qPCR.

FIG. 4H illustrates the effects of various inhibitors and concentrations thereof on the mRNA expression levels in ATDC5 cells. In FIG. 4H, the left bar for each concentration represents aggrecan, and the right bar represents type II collagen.

FIG. 4I illustrates the effects of various inhibitors and concentrations thereof on the mRNA expression levels in primary human chondrocytes. In FIG. 4I, the left bar for each concentration represents aggrecan, and the right bar represents type II collagen.

FIG. 5A is a bar graph showing the effect of different concentrations of ugonin P on the expression level of miR-3074-5p in ATDC5 cells, with a treatment time of 24 hours.

FIG. 5B is a bar graph showing the effect of different concentrations of ugonin P on the expression level of miR-3074-5p in primary human chondrocytes, with a treatment time of 24 hours.

FIG. 5C is a bar graph showing the effects of different types of stimuli on the mRNA expression levels of aggrecan and type II collagen in ATDC5 cells.

FIG. 5D is a bar graph showing the effects of different types of stimuli on the mRNA expression levels of aggrecan and type II collagen in primary human chondrocytes.

FIG. 5E is a representative Western blot image showing that the enhancing effect of ugonin P on aggrecan or type II collagen in ATDC5 cells is affected by NC mimics or miR-3074-5p mimics.

FIG. 5F quantitatively represents, via a bar graph, that the enhancing effect of ugonin P on aggrecan or type II collagen in ATDC5 cells is affected by NC mimics or miR-3074-5p mimics.

FIG. 5G is a representative Western blot image showing that the enhancing effect of ugonin P on aggrecan or type II collagen in primary human chondrocytes is affected by NC mimics or miR-3074-5p mimics.

FIG. 5H quantitatively represents, via a bar graph, that the enhancing effect of ugonin P on aggrecan or type II collagen in primary human chondrocytes is affected by NC mimics or miR-3074-5p mimics.

FIG. 5I shows Alcian blue staining results illustrating that the enhancing effect of ugonin P on aggrecan or type II collagen in ATDC5 cells is affected by NC mimics or miR-3074-5p mimics, with a treatment time of 2 weeks.

FIG. 5J is the quantitative result of FIG. 5I, presented as absorbance values at a wavelength of 620nm.

FIG. 6A shows the differential gene expression between ATDC5 cells and ATDC5 cells treated with ugonin P.

FIG. 6B shows differential gene expression after ugonin P treatment through GO enrichment analysis, where the horizontal axis represents the enrichment level and the vertical axis lists pathways with higher enrichment, including glycosaminoglycan biosynthesis-heparan sulfate/heparin and ECM-receptor interaction.

FIG. 6C shows differential gene expression after ugonin P treatment through KEGG analysis, where arrows point to, from top to bottom: collagen-containing extracellular matrix, extracellular matrix, collagen fibril organization, activation of MAPK activity, and extracellular matrix organization.

FIG. 6D shows differential gene expression after ugonin P treatment through IPA pathway analysis, where the arrow points to the osteoarthritis pathway.

FIG. 7A is a pathway relationship diagram from IPA pathway analysis.

FIG. 7B is a representative Western blot image showing the expression of related proteins in ATDC5 cells after treatment with ugonin P, where the molecular weight unit of the proteins is kilodaltons (kDa).

FIG. 7C is the quantitative result of FIG. 7B, showing changes in the phosphorylation ratios of proteins in ATDC5 cells after treatment with ugonin P, where from left to right for each treatment time are pERK/ERK, pJNK/JNK, and p-p38/p38.

FIG. 8A to 8L illustrate that the effects of ugonin P on aggrecan and type II collagen in ATDC cells or primary human cells are regulated via the MAPK pathway. FIG. 8A to 8D include quantitative mRNA plots, and FIG. 8E to 8L include Western blot results and quantitative plots, where the signaling pathway of ugonin P is verified by adding inhibitors or siRNA. The inhibitors include FR180204 (ERK inhibitor), SP600125 (JNK inhibitor), and SB203580 (p38 inhibitor), and the siRNA includes ERK siRNA, JNK siRNA, and p38 siRNA.

### DETAILED DESCRIPTION

The technical effects of the present invention are further described below with the embodiments and the drawings.

In some embodiments, the present invention provides a use of a ugonin compound in the manufacture of a composition for preventing or treating a joint-related disease, wherein the ugonin compound enhances chondrogenesis; it is understood that the chondrogenesis includes cell proliferation, cell differentiation, and matrix generation.

It is understood that the ugonin compound is a flavonoid compound, which is a natural product and can be isolated from a plant; however, the ugonin compound is not limited to being obtained by artificial synthesis, biosynthesis, or any combination of the aforementioned methods. In some embodiments, the ugonin compound is derived from the genus Helminthostachys, and more specifically, from Helminthostachys zeylanica and its parts including, but not limited to, roots, rhizomes, stems, or leaves.

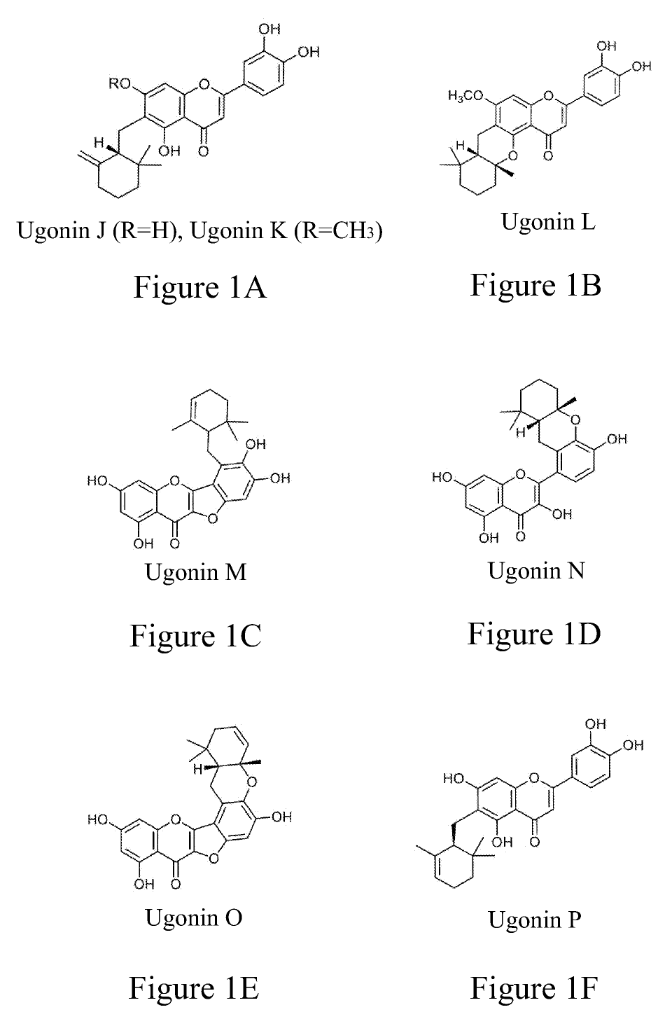

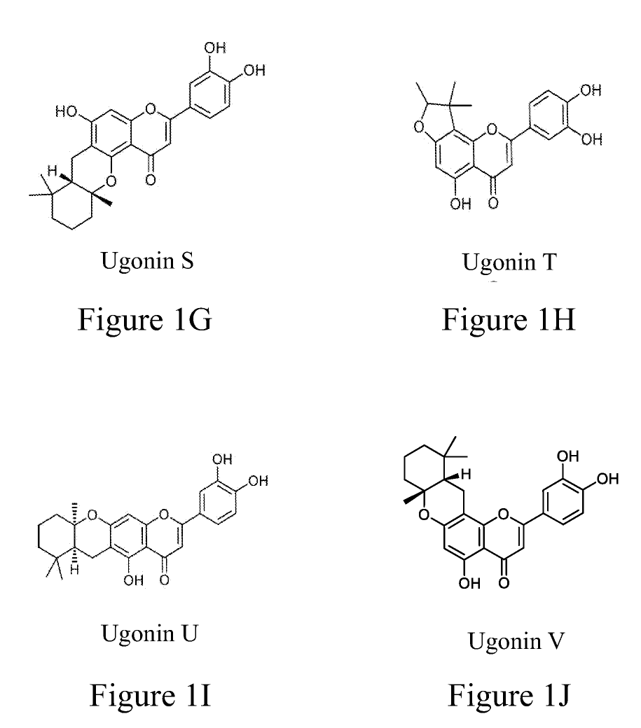

Referring to FIGS. 1A to 1J, in some embodiments, the ugonin compound is selected from one or more of the following: ugonin J, ugonin K, ugonin L, ugonin M, ugonin N, ugonin O, ugonin P, ugonin S, ugonin T, ugonin U, and ugonin V. Among them, the molecular formula of ugonin J is C₂₅H₂₆O₆ with a molecular weight of 422.5g/mol; ugonin K is C₂₆H₂₈O₆ (436.5g/mol); ugonin L is C₂₆H₂₈O₆ (436.5g/mol); ugonin M is C₂₅H₂₄O₇ (436.5g/mol); ugonin N is C₂₅H₂₆O₇ (438.5g/mol); ugonin O is C₂₅H₂₂O₇ (434.4g/mol); ugonin P is C₂₅H₂₆O₆ (422.5g/mol); ugonin S is C₂₅H₂₆O₆ (422.5g/mol); and ugonin T is C₂₀H₁₈O₆ (354.4g/mol).

It is also understood that the joint-related disease includes conditions that result in pain, stiffness, or reduction in range of motion. The joint may be located in the head, neck, spine, shoulder, elbow, hand, hip, knee, or foot of the human body. The joint includes bone, articular cartilage, joint capsule, synovial fluid, ligaments, tendons, and bursae.

In some embodiments, the joint-related disease is selected from one or more of the following: arthritis, chondrodysplasia, chondral injury, chondromalacia patella, chondritis, and chondrolysis, without limitation. The arthritis is selected from one or more of the following: osteoarthritis (OA), rheumatoid arthritis (RA), metabolic arthritis, psoriatic arthritis, and ankylosing spondylitis, without limitation. It is further understood that osteoarthritis may also be referred to as degenerative arthritis or hypertrophic arthritis; metabolic arthritis is commonly known as gout.

It can be appreciated that the cartilage of patients suffering from such joint-related diseases may require treatment or repair. Various causes may lead to the need for cartilage treatment or repair; therefore, the joint-related disease may be caused by immunological factors, infectious factors, aging, genetic factors, metabolic factors, pharmaceutical/chemical factors, mechanical injury, or any combination thereof, without limitation.

Further, in some embodiments, the ugonin compound promotes a cartilage-forming cell to generate an extracellular matrix (ECM) to enhance chondrogenesis. It is understood that the cartilage-forming cell includes chondroblast and chondrocyte, which can generate the ECM including the cartilage matrix. In human physiology, chondrocytes are embedded in the cartilage matrix and maintain cartilage function through proliferation or production of the cartilage matrix.

Specifically, the extracellular matrix includes collagen, glycosaminoglycan, and proteoglycan. The collagen includes type I collagen (COL1) and type II collagen (COL2); the glycosaminoglycan includes chondroitin sulfate or hyaluronic acid; and the proteoglycan includes aggrecan (ACAN) but are not limited thereto.

In some embodiments, the ugonin compound reaches an effective concentration in a biofluid of a subject to promote the cartilage-forming cells in the joints of the subject to generate the extracellular matrix and enhance chondrogenesis, wherein the biofluid is in contact with the cartilage-forming cells.

It is further understood that the biofluid includes blood, plasma, interstitial fluid, lymph fluid, and synovial fluid.

In some embodiments, the effective concentration is between 0.1 and 100μM. In some embodiments, the effective concentration is between 1 and 90μM, and the extracellular matrix is the proteoglycan. In some embodiments, the effective concentration is between 0.3 and 30μM, and the extracellular matrix is collagen.

In some embodiments, the effective concentration is between 0.035 and 45mg/L. In some embodiments, the effective concentration is between 0.35 and 40.5mg/L, and the extracellular matrix is the proteoglycan. In some embodiments, the effective concentration is between 0.105 and 13.5mg/L, and the extracellular matrix is collagen. It is understood that the molecular weight of the ugonin compound is selected between 350 and 450g/mol.

Further, in some embodiments where the biofluid is plasma, the total volume of plasma in the subject should be considered for the conversion of the effective dose. Similarly, where the biofluid is synovial fluid, the total volume of synovial fluid in the joint should be considered; for example, if the knee synovial fluid volume is 3mL and the effective concentration is 10mg/L, 0.03mg of the ugonin compound should be provided in the knee synovial fluid.

In some embodiments, the ugonin compound suppresses expression of a microRNA in a cartilage-forming cell, and activates a signaling pathway to enhance chondrogenesis, wherein the microRNA includes miR-3074-5p, and the signaling pathway includes the mitogen-activated protein kinase (MAPK) pathway.

In some embodiments, the composition comprises a non-zero mass percentage of the ugonin compound to obtain its active effect. The composition can be administered to a subject in a dosage form and via an administration route in an effective amount. The dosage form can be solid, liquid, or a mixture thereof, such as powders, tablets, capsules, or emulsions. It can also be used externally on the body surface via spray or internally via inhalation for systemic circulation.

In some embodiments, the subject includes mammals, such as humans (Homo sapiens), cats, or dogs, without limitation. In some embodiments, the administration route includes injection, oral administration, or topical application, wherein the injection includes subcutaneous injection, intravenous injection, or intramuscular injection, and the topical application is performed on skin or mucosa, thereby the active effect of the ugonin compound is manifested in the joints of the subject.

In some embodiments, the ugonin compound can be used to prevent the onset or progression of joint-related diseases. For example, some agents, such as anesthetics or anti-inflammatory drugs, may exert toxicity or negative impacts on cartilage during use. Based on the property of the ugonin compound in promoting chondrogenesis, it can be used in combination with these cartilage-toxic drugs to offset or counteract the side effects caused to the cartilage.

In some embodiments, the ugonin compound can be used to prevent or treat joint-related diseases. For example, some athletes may experience cartilage wear during high-intensity exercise. Once the wear reaches a certain threshold, symptoms such as pain, stiffness, or reduced range of motion may occur, which can be classified as a disease. Based on its chondrogenesis-promoting property, athletes can ingest the ugonin compound intermittently or continuously before the wear reaches the threshold to offset or counteract the wear from high-intensity exercise, thereby producing a preventive effect. Alternatively, even after the wear reaches the threshold, cartilage regeneration achieved through intermittent or continuous ingestion of the ugonin compound can produce a therapeutic effect.

The technical effects of the present invention are further illustrated through specific examples below.

Regarding the preparation of ugonin P, it was prepared according to the method provided in the literature "Anti-inflammatory flavonoids from the rhizomes of Helminthostachys zeylanica" published in 2009, which should be understood by those having ordinary skill in the art and will not be further described here.

For cell experiments, the mouse chondrogenic cell line (ATDC5) was used and cultured in a medium consisting of DMEM and Ham's F12 at a 1:1 volume ratio. The medium may further include 5% fetal bovine serum (FBS), 100 U/mL penicillin, and 100 μg/mL streptomycin. Cell culture was conducted at 37°C in a 5% CO2 atmosphere. Regarding primary human chondrocytes, they were obtained from cartilage samples from total knee arthroplasty. After appropriate cutting, the cartilage samples were treated with 0.1% hyaluronidase and 0.2% collagenase for 30 and 60 minutes, respectively, and then passed through a 70 μm nylon cell strainer to obtain primary human chondrocytes. These cells were cultured in DMEM containing 20 mM HEPES, 10% FBS, 2 mM L-glutamine, 100 U/mL penicillin, and 100 μg/mL streptomycin.

For bioinformatics analysis, the dataset GSE55235 from the Gene Expression Omnibus (GEO) was used to analyze aggrecan and type II collagen. QIAGEN^{®} Ingenuity Pathway Analysis (IPA) was used to analyze canonical signaling pathways. The open-source software miRWalk 3.0 was used to analyze the potential binding of microRNA (miRNA) to aggrecan and type II collagen. GEO databases (GSE143514, GSE175962, GSE124373) were used to analyze miRNA expression levels via sequencing.

The operation of the miRWalk 3.0 database includes: (1) Target mining: select the "Gene" option and species such as "mouse" or "human," enter Entrez gene IDs (e.g., 176 for ACAN or 1280 for COL2A1), and click "submit" and "process." (2) Database selection: the user is directed to a new tab with databases such as TargetScan, miRDB, and miRTarBase. The 3'UTR option can be used to refine search criteria, generating miRNAs predicted to bind to the 3'UTR regions of ACAN and COL2A1; higher scores indicate higher consistency across databases.

In the MTT assay, cells were treated with different doses of ugonin P (including a 0 dose) for one day, followed by the addition of MTT solution (0.5mg/mL) and DMSO dissolution. Absorbance at 570 nm was measured using a BioTek® reader.

In quantitative real-time PCR (qPCR) assays, RNA was extracted using the TRIzol method, followed by cDNA synthesis. miRNA expression was measured using a StepOnePlus™ Real-Time PCR System. TRIzol reagent was purchased from MDBio, Inc. (Taipei, Taiwan); the M-MLV Reverse Transcriptase kit (Thermo Fisher Scientific) was used for cDNA synthesis; and the Mir-X™ miRNA First-Strand Synthesis kit (Terra Bella Avenue, Mountain View, CA, USA) was used for miRNA measurement. Glyceraldehyde 3-phosphate dehydrogenase (GAPDH) and U6 were used as housekeeping genes for internal normalization of mRNA and miRNA expression, respectively.

In RNA sequencing, a sample library was established, and differential indicators were analyzed using an Illumina (HiSeq, Novaseq) or MGI2000 sequencer for paired-end sequencing. Differential expression analysis was performed using the DESeq2 Bioconductor package with thresholds of |log2 fold change (FC)| > 2 and adjusted p-value < 0.05. The Kyoto Encyclopedia of Genes and Genomes (KEGG) was used as the database.

In Western blot analysis, cells were lysed using lysis buffer containing protease inhibitors to obtain protein samples of equal concentration. After SDS-PAGE, proteins were transferred to a PVDF membrane, which was then blocked with non-fat milk and Tris buffer and incubated with primary antibodies overnight at 4°C. After washing with TBST, the membrane was incubated with HRP-conjugated secondary antibodies for 1 hour at room temperature. Finally, ECL reagent was used for autoradiography to detect target proteins.

Alcian Blue staining targets proteoglycans, mucins, and other cartilage components. In experiments involving Alcian Blue staining, differentiation medium was added to ATDC5 cells to induce differentiation. The medium included 1% insulin-transferrin-selenium solution (1mg/mL insulin, 0.55mg/mL transferrin, and 0.67mg/mL sodium selenite) and 50mg/mL L-ascorbic acid 2-phosphate. Cells underwent a 14-day differentiation period with medium changes every 2 to 3 days. After differentiation, cells were fixed with 4% formaldehyde and 1% Alcian Blue 8GX, washed with deionized water, and treated with 6mol/L guanidine for 2 hours. Finally, absorbance at 620 nm was detected and images were captured.

Cell transfection involved seeding 5×10⁵ cells in 6-well plates and using Lipofectamine^{®}2000 to transfect miRNA, miRNA inhibitors, or pharmacological siRNA.

Statistical analysis was performed using GraphPad Prism 8.2. One-way ANOVA with Bonferroni’s post hoc test was used for groups of three or more, and two-way ANOVA was used for two-factor relationships. Results are presented as mean ± standard deviation, with p < 0.05 considered statistically significant.

Example 1: aggrecan and type II collagen (COL2) expression levels are downregulated in OA and RA patients.

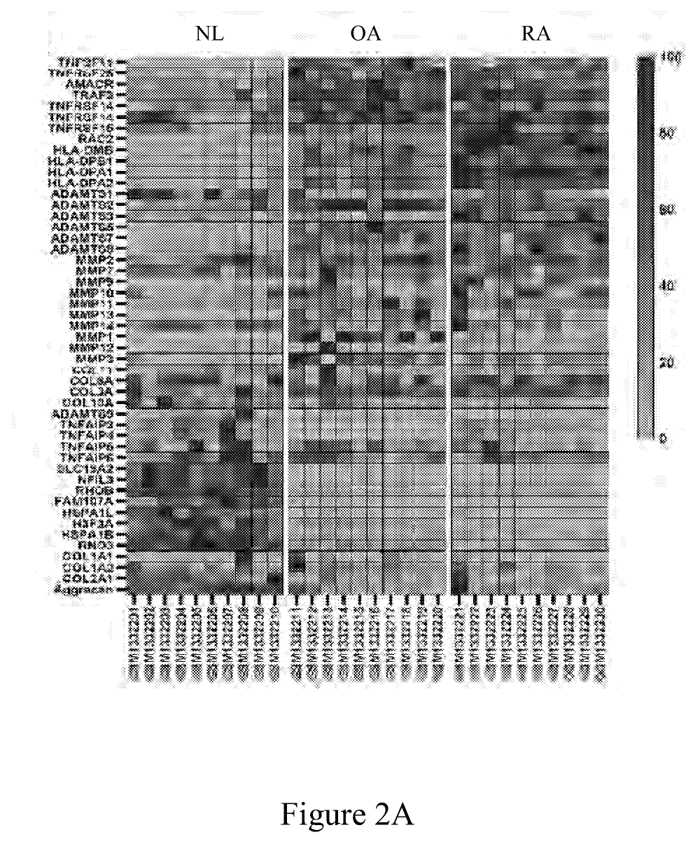

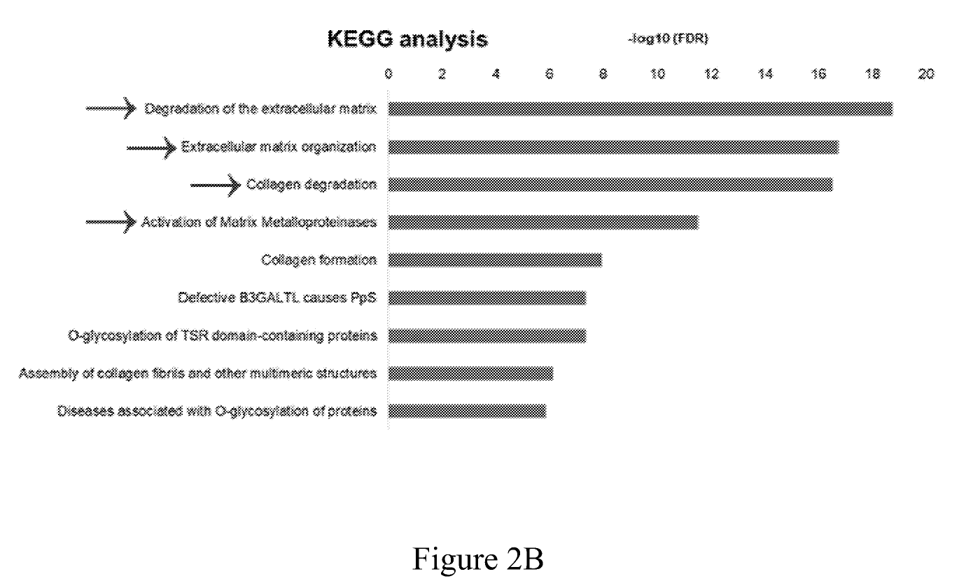

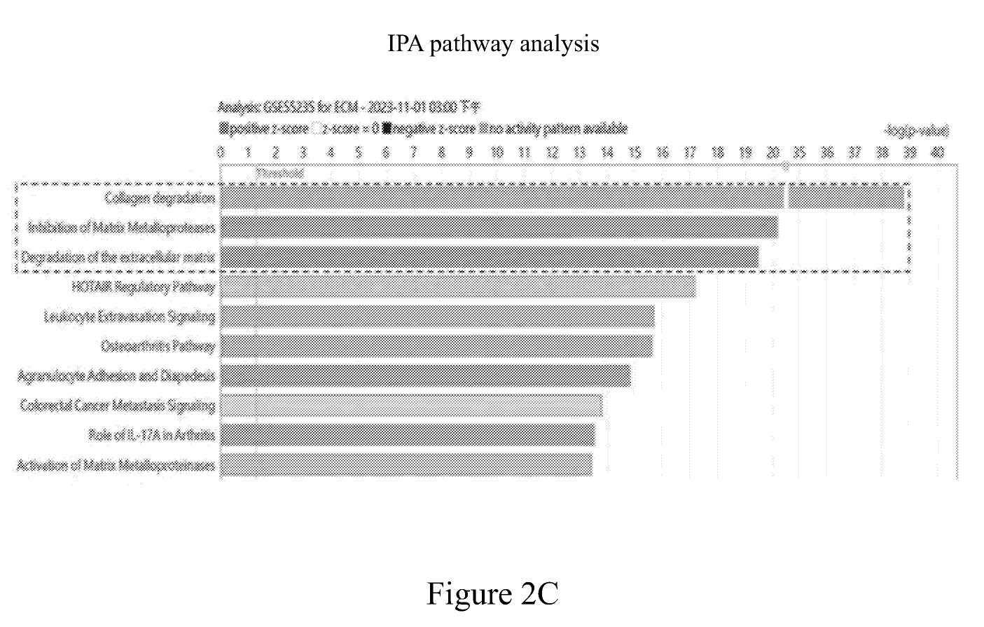

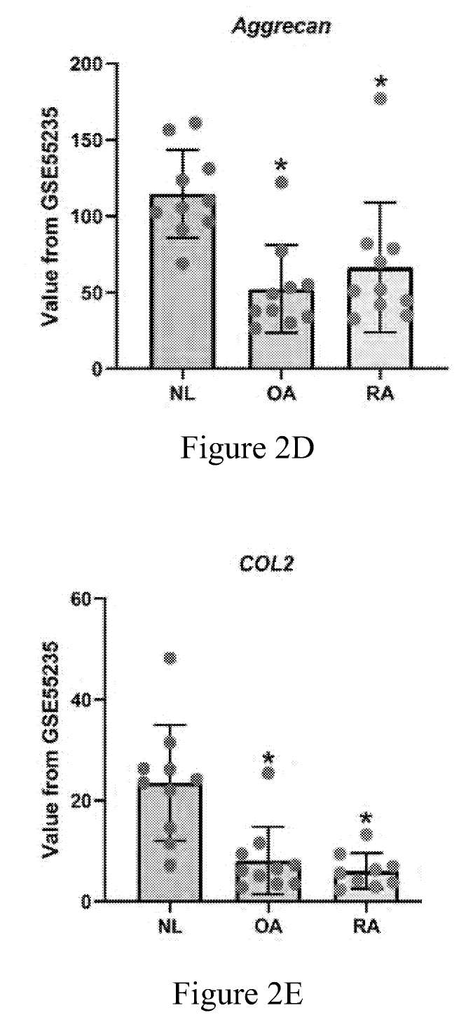

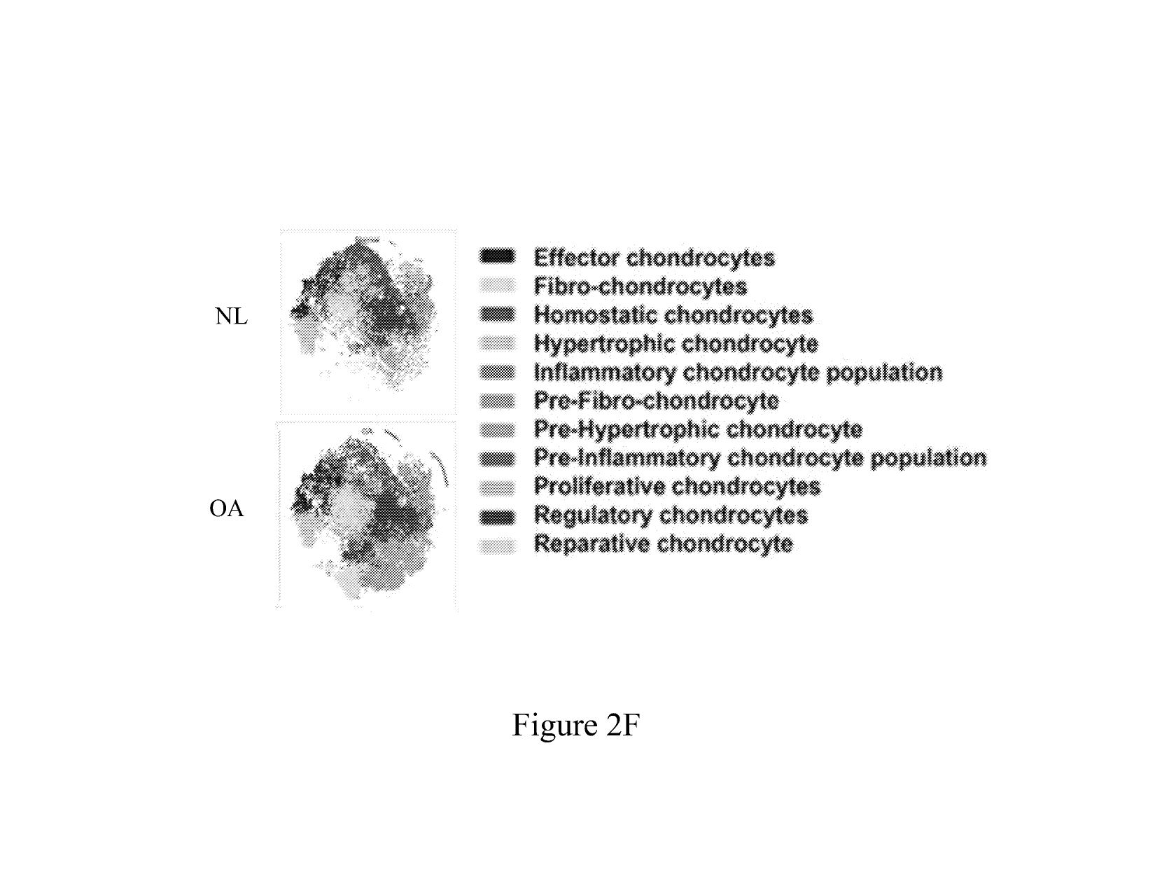

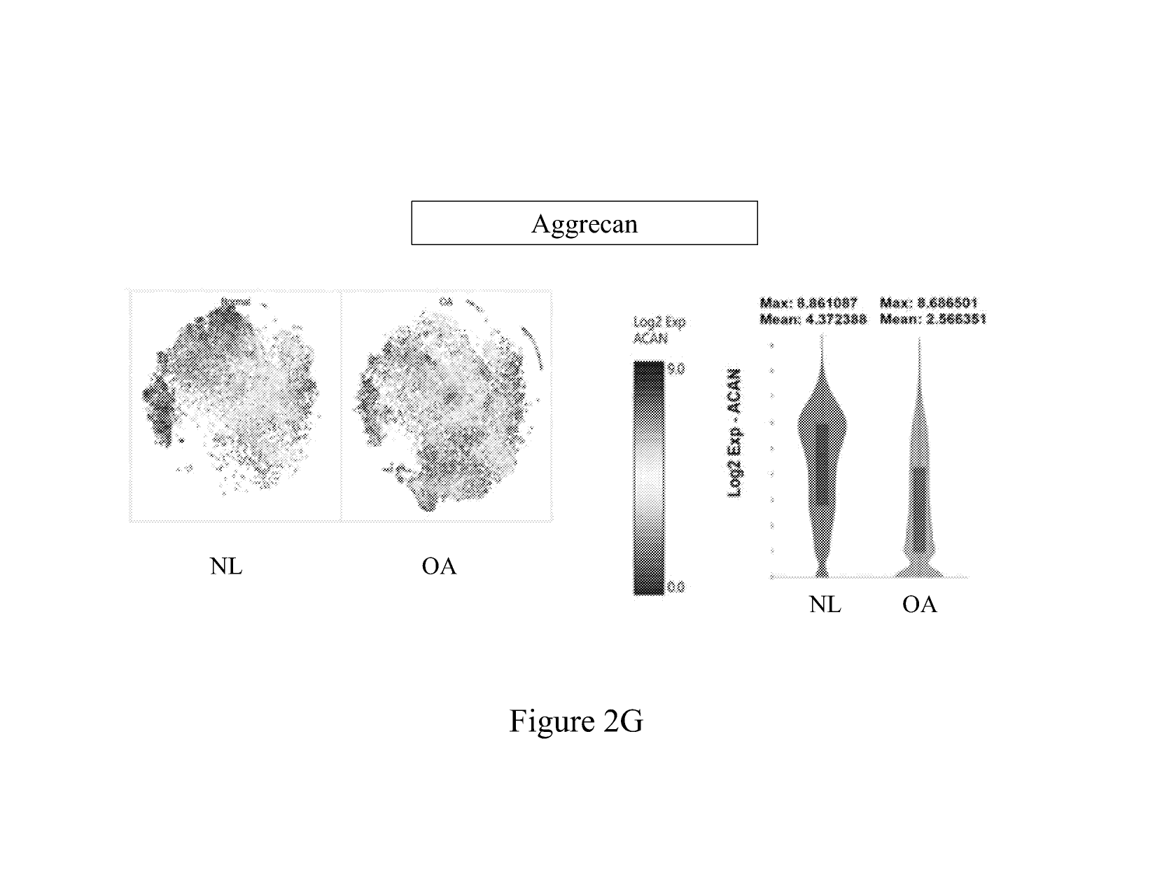

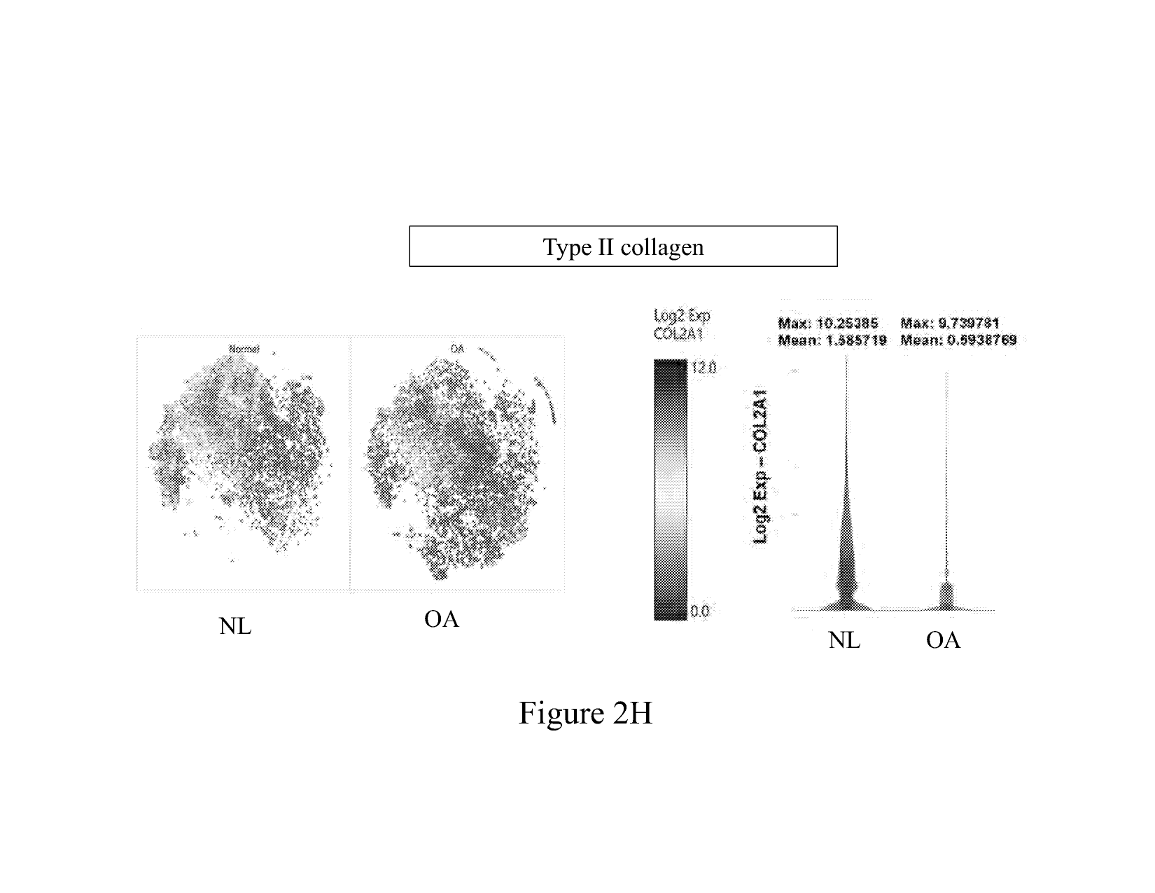

Synovial inflammation, cartilage degradation, and bone destruction are common features in OA and RA patients. To confirm these in clinical patients, GEO database analysis was performed. Referring to FIG. 2A, the GSE55235 dataset analysis showed differential gene expression between RA patients and healthy subjects. Further, referring to FIGS. 2B and 2C, KEGG and IPA analyses revealed that biological functions such as collagen degradation, ECM destruction, and matrix metalloproteinase activation were more prevalent in OA and RA patients. Referring to FIGS. 2D and 2E, cartilage components including aggrecan and type II collagen showed lower expression in OA and RA patients compared to healthy subjects. These results indicate a reduction in ECM and cartilage components in OA and RA patients. To further understand the roles played by aggrecan and type II collagen in arthritis patients, this embodiment further analyzed single-cell RNA sequencing data (GSE55460). Please refer to FIG. 2F, which illustrates the cellular composition of healthy subjects and OA patients. Furthermore, FIG. 2G and FIG. 2H respectively show the differences in the expression levels of aggrecan and type II collagen within the cartilage tissues of healthy subjects and OA patients. These results clearly demonstrate that the degradation of the extracellular matrix (ECM)—specifically the reduction of cartilage matrix components including aggrecan and type II collagen—is widespread among arthritis patients.

Example 2: ugonin P treatment increases aggrecan and type II collagen expression, thereby enhancing chondrogenesis.

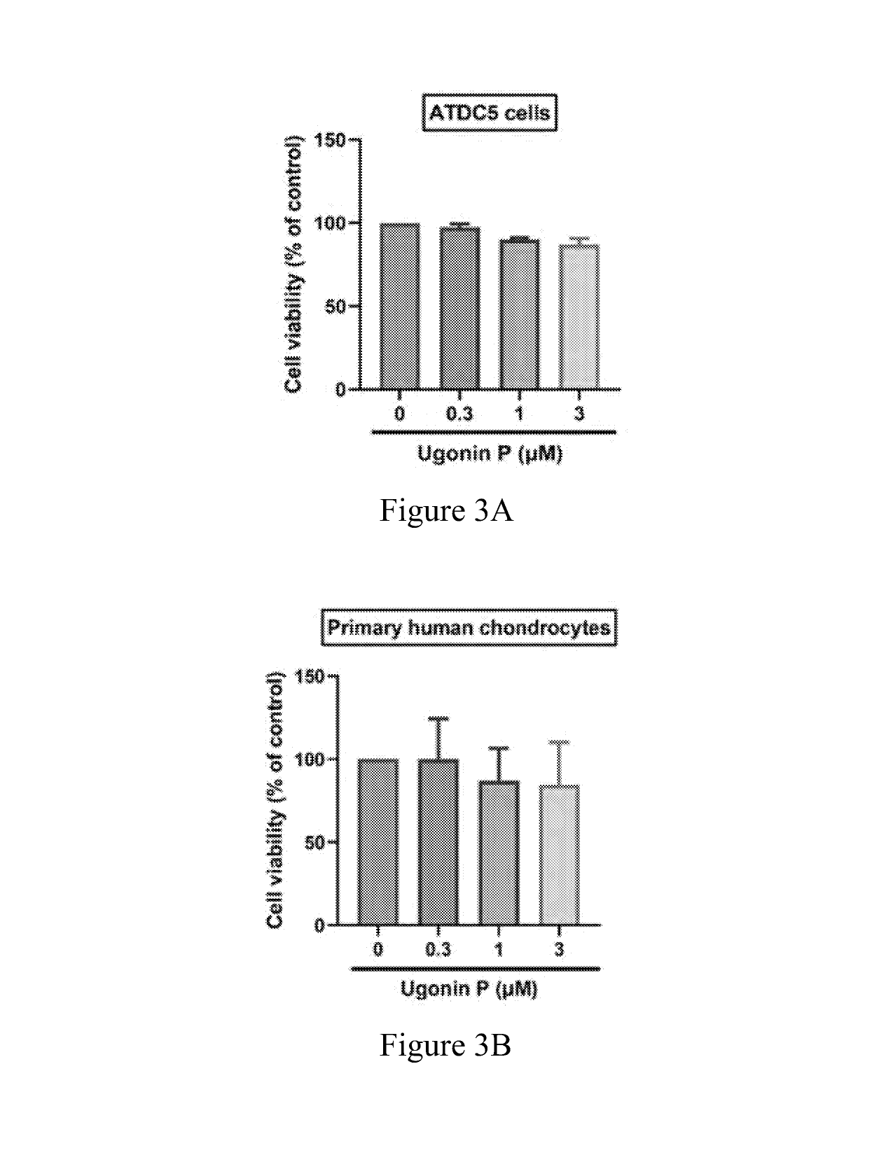

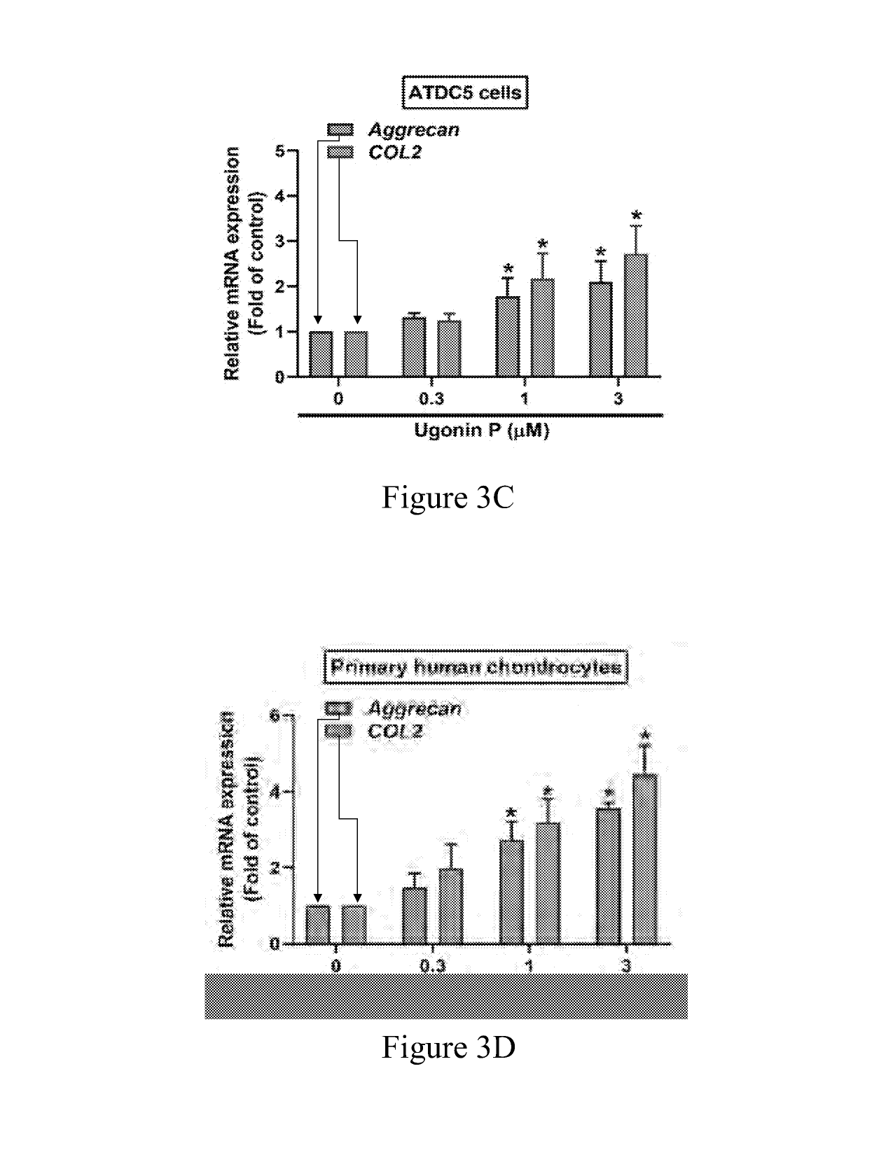

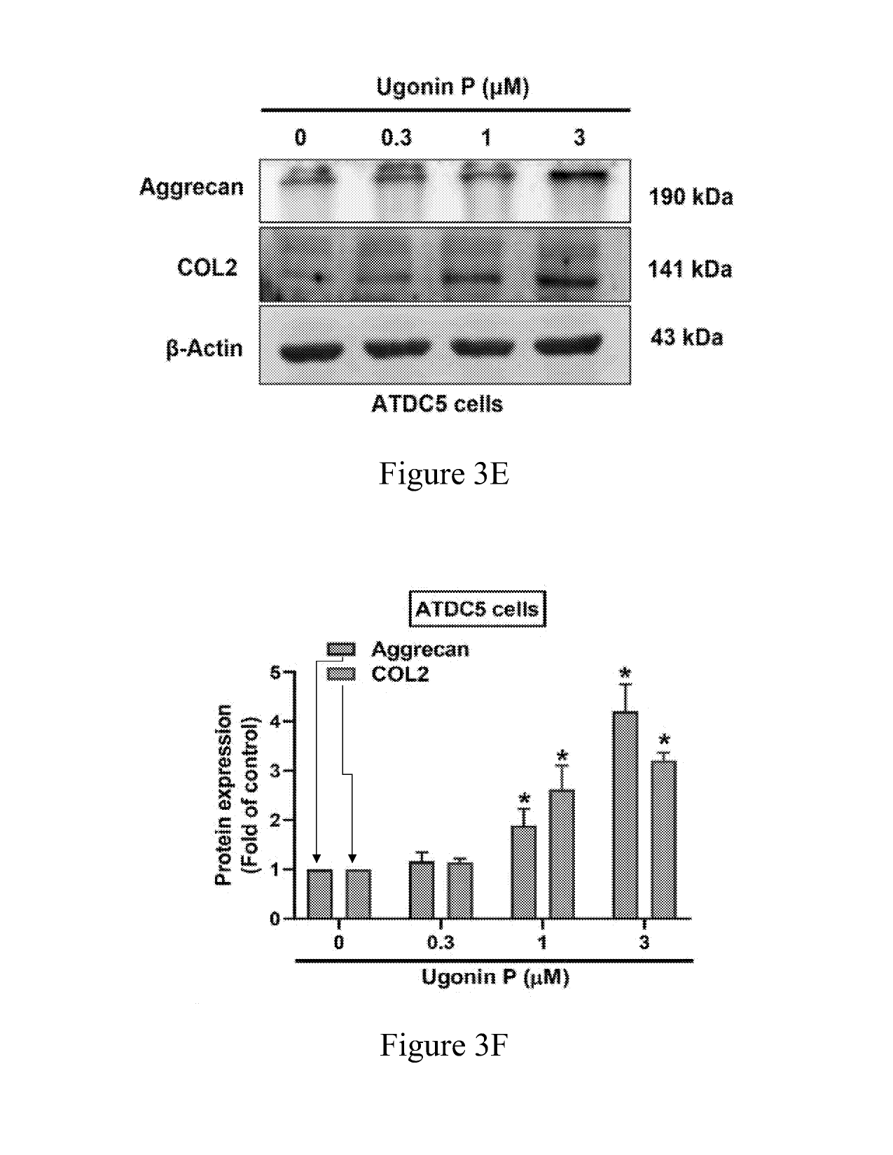

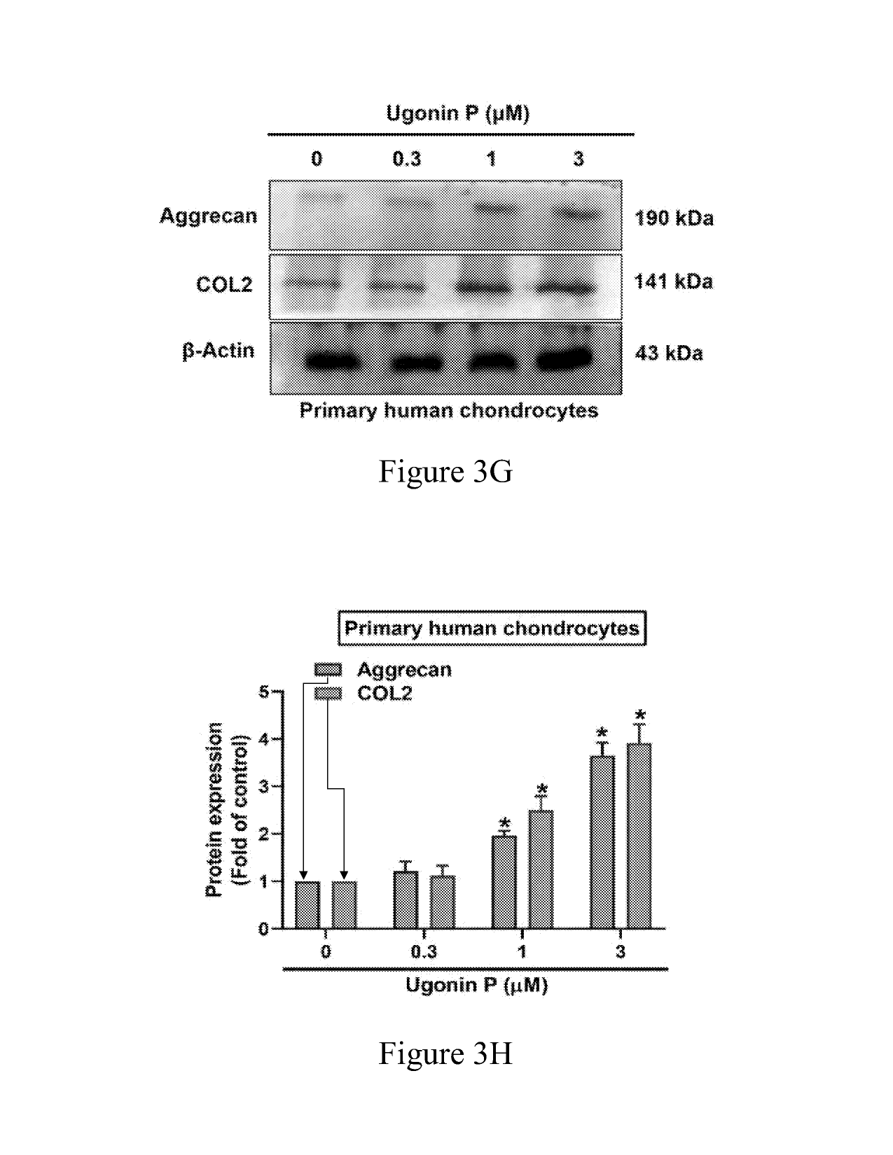

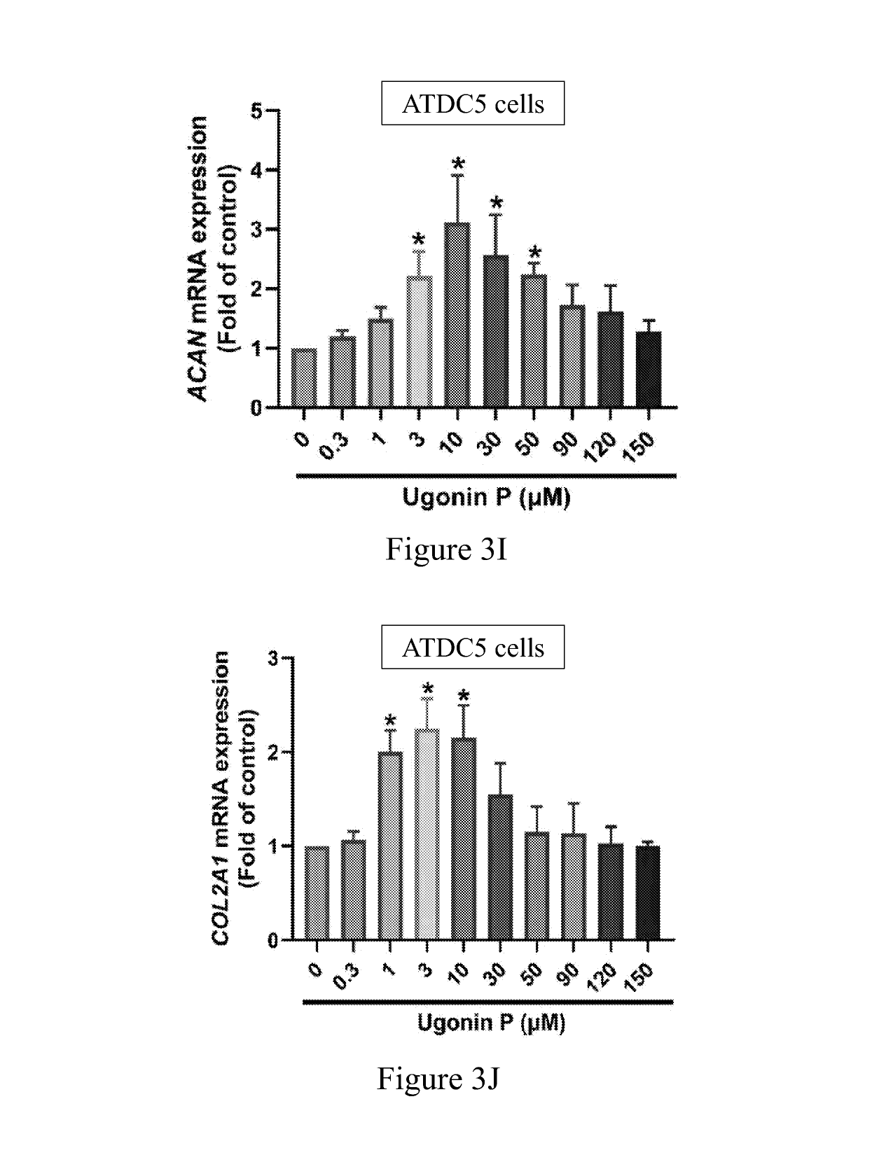

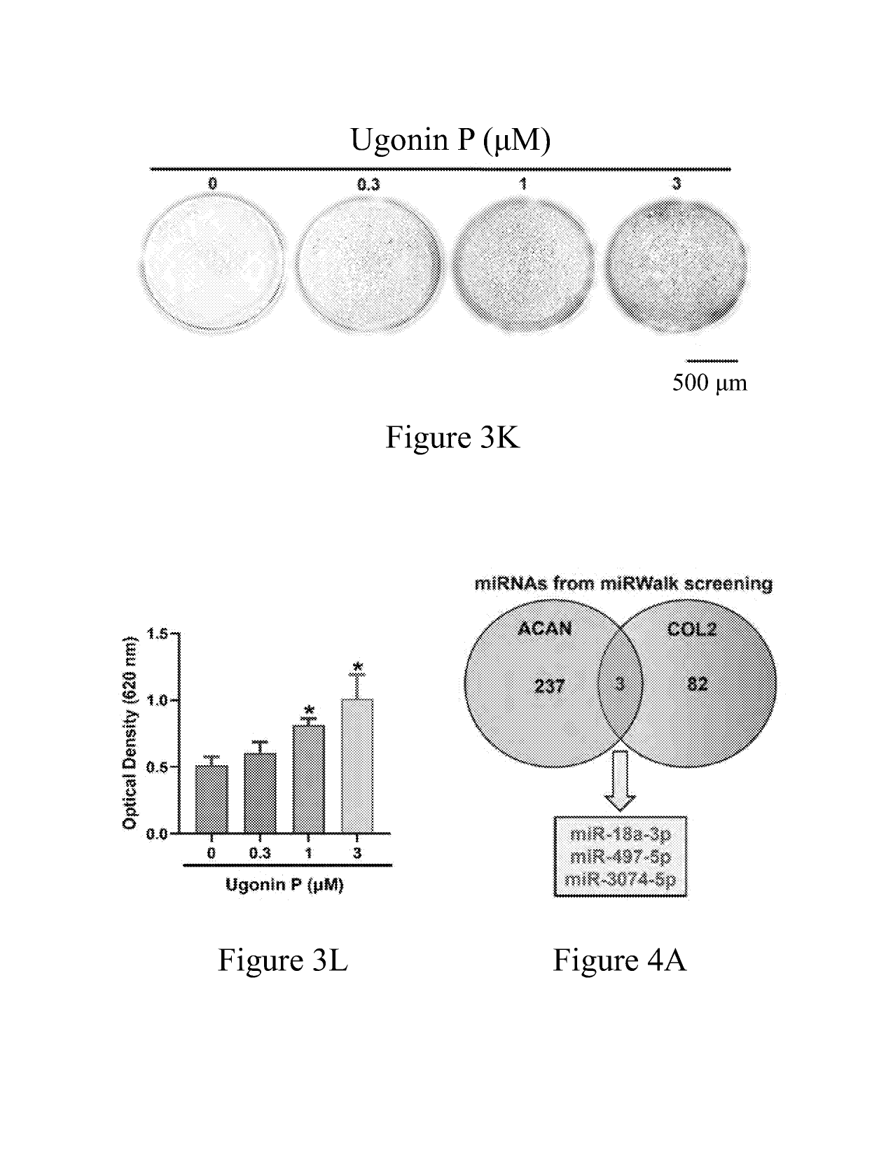

Referring to FIG. 1F, which shows the chemical structure of ugonin P. Regarding chondrocytes, this embodiment was conducted using the ATDC5 chondrogenic cell line and primary human chondrocytes. Referring to FIG. 3A and FIG. 3B, the results were obtained by treating the ATDC5 chondrogenic cells and the primary human chondrocytes with ugonin P, respectively, and analyzing the cell viability using an MTT assay to observe whether ugonin P exhibits cytotoxicity. The results show that within a relevant dose range, ugonin P does not affect the viability of either cell type. Next, referring to FIGS. 3C to 3H, it can be observed that after treating the ATDC5 chondrogenic cells and the primary human chondrocytes with ugonin P, the messenger RNA (mRNA) and protein expression levels of both aggrecan and type II collagen are significantly increased. Further referring to FIG. 3I and FIG. 3J, which respectively represent the effects of ugonin P on the expression of aggrecan and type II collagen in ATDC5 cells, it can be found that ugonin P at a dose between 3μM and 50μM is able to enhance the mRNA expression of aggrecan, and ugonin P at a dose between 1μM and 10μM is able to enhance the mRNA expression of type II collagen.

Referring to FIG. 3K and FIG. 3L, the ATDC5 cells were cultured in differentiation medium with ugonin P for 14 days. As a result, Alcian Blue staining showed a corresponding increase in cartilaginous matrices as the concentration of ugonin P administered increased. Accordingly, it is understood that ugonin P promotes the expression of aggrecan and type II collagen in chondrocytes, thereby enhancing the promotion of chondrogenesis.

Example 3: proof that miR-3074-5p regulates ugonin P-enhanced aggrecan and type II collagen expression and chondrogenesis.

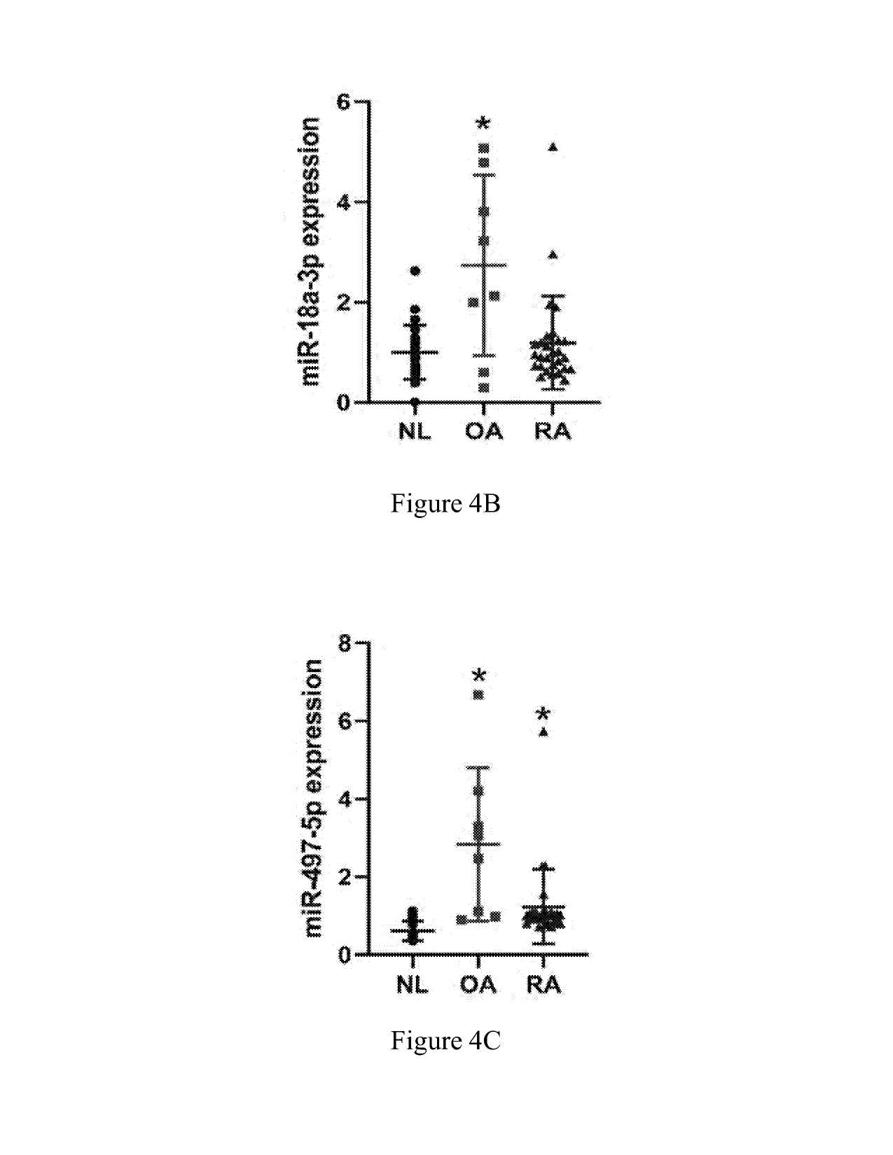

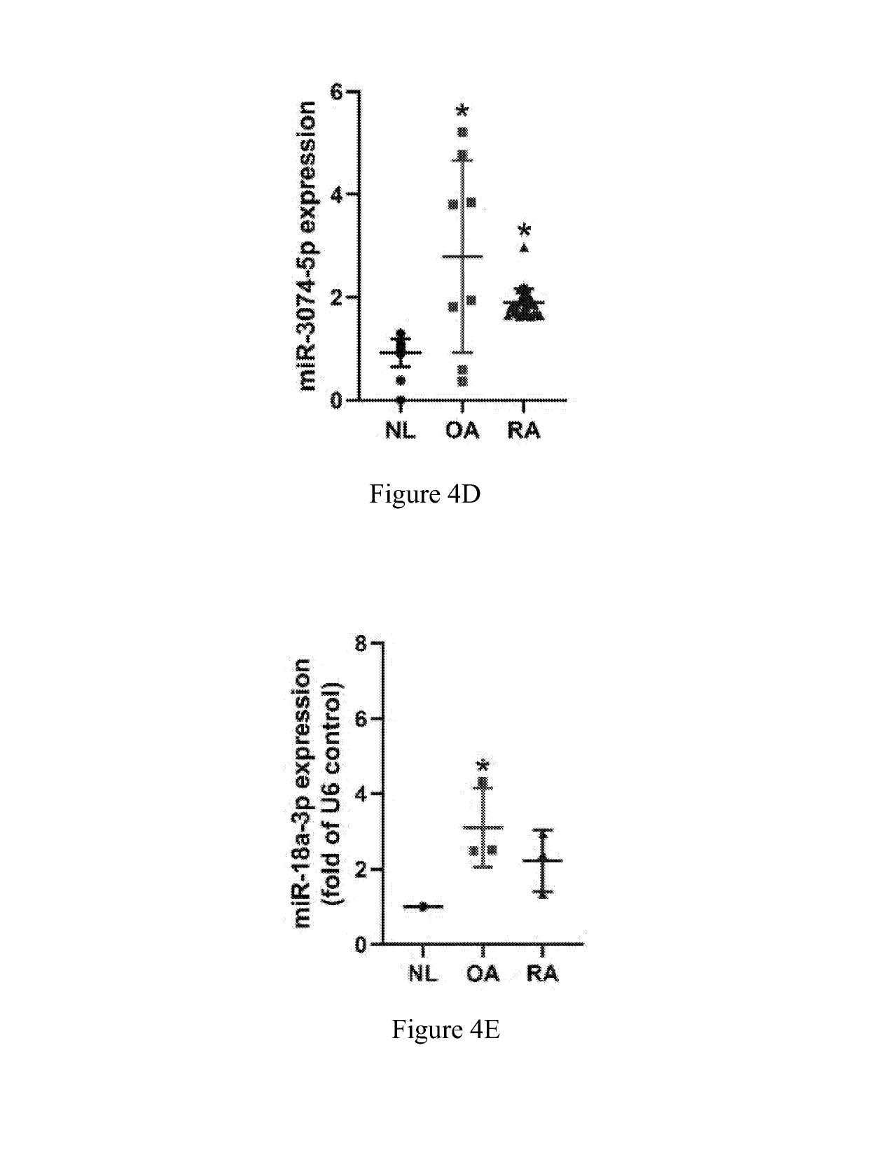

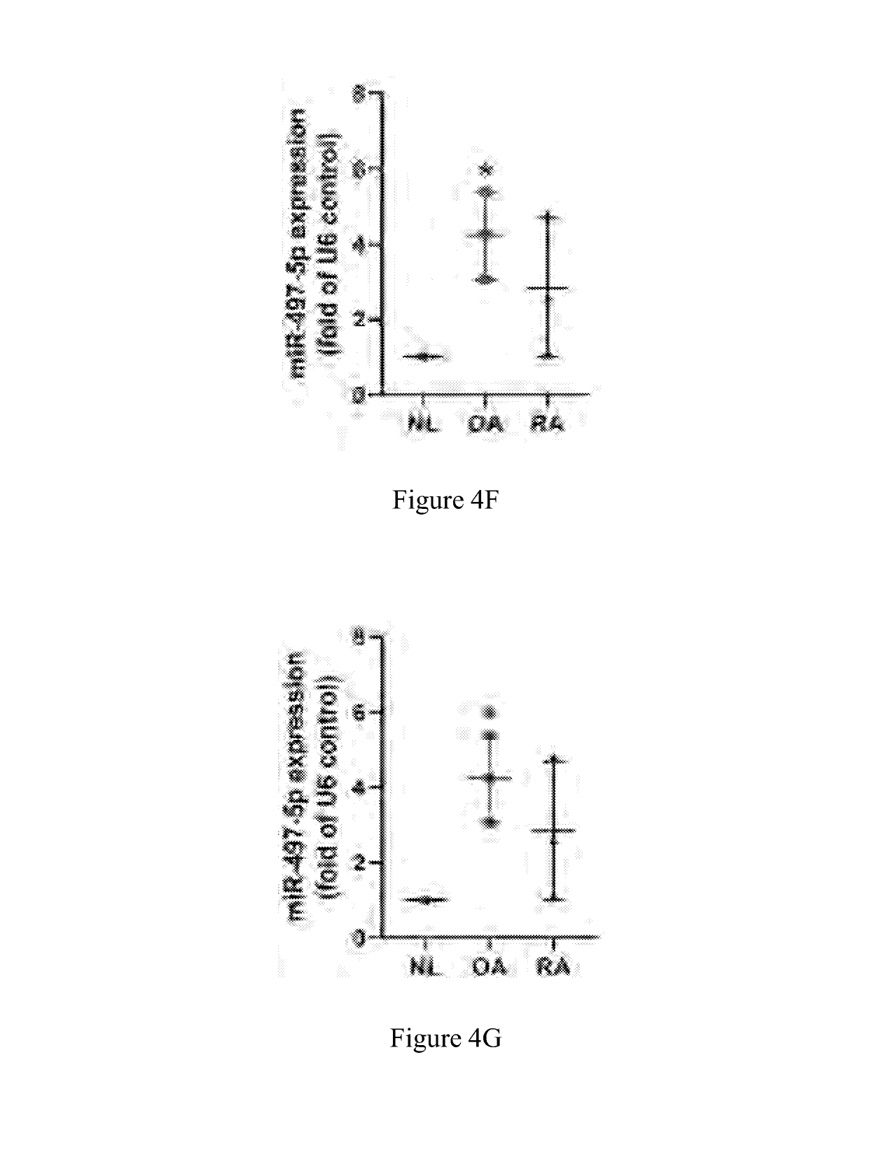

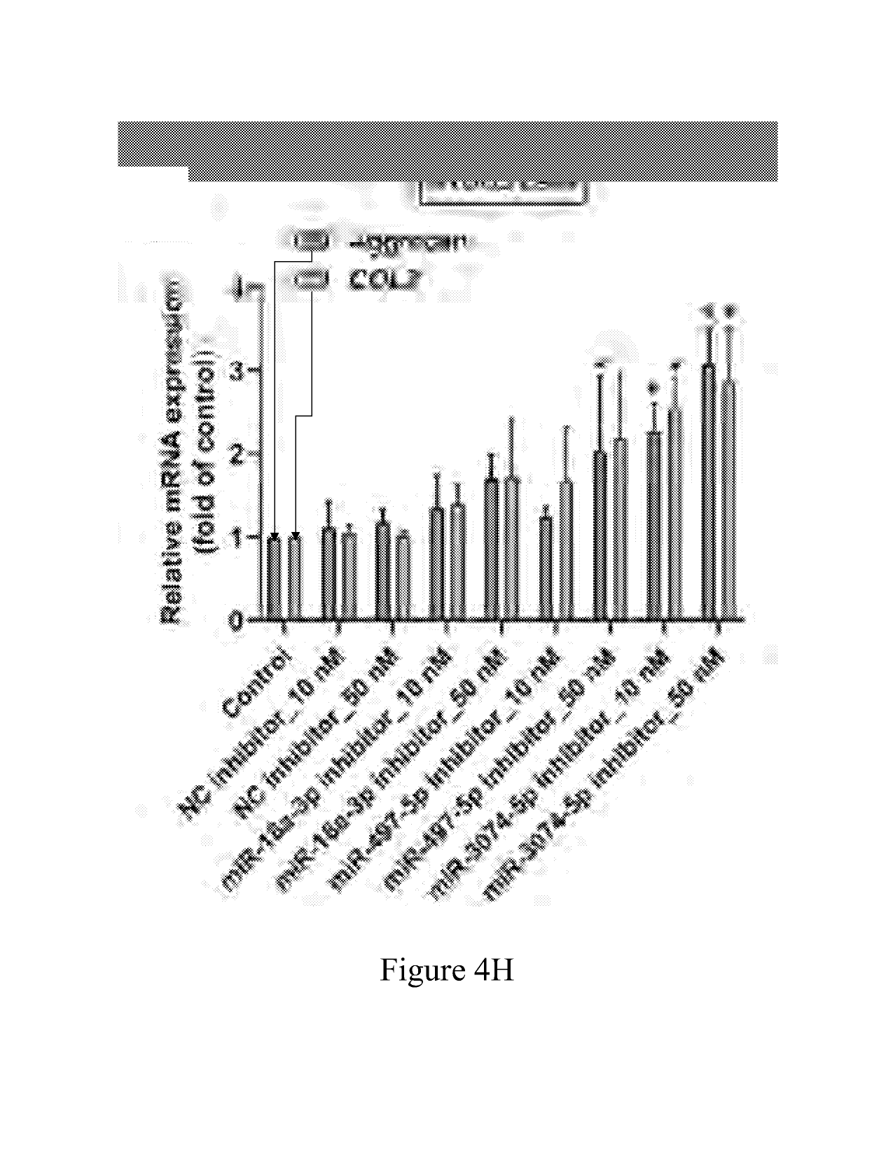

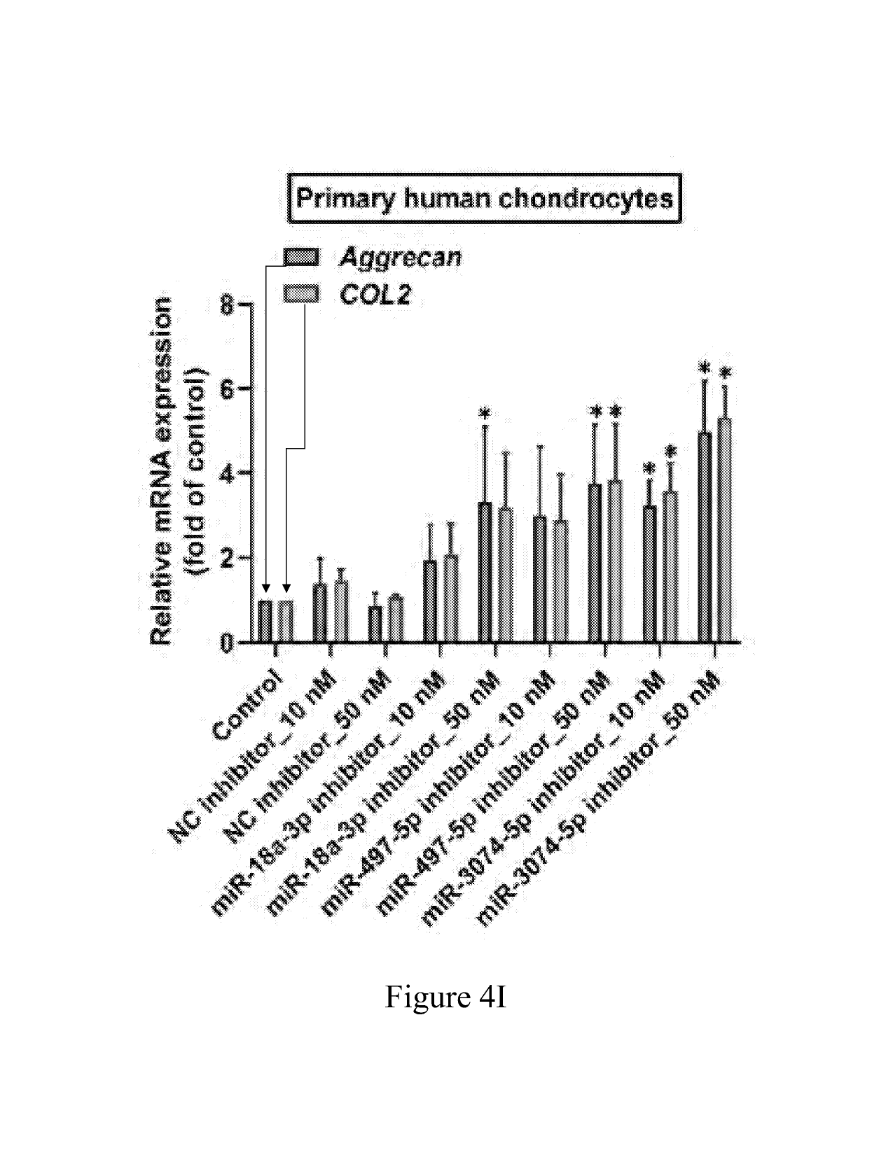

Referring to FIG. 4A, in this embodiment, miRNA bioinformatics software was employed to identify microRNAs (miRNAs) that directly interact with the 3' untranslated regions (3' UTRs) of aggrecan and type II collagen. Three primary candidates were identified: miR-18a-3p, miR-497-5p, and miR-3074-5p. To explore the clinical correlation of these miRNAs, on one hand, although high-throughput gene expression database analysis showed that miR-497-5p and miR-3074-5p were significantly upregulated in OA and RA patients (as shown in FIGS. 4B to 4D); on the other hand, clinical data indicated that only miR-3074-5p—excluding miR-18a-3p and miR-497-5p—was increased in OA and RA patients (as shown in FIGS. 4E to 4G). Referring to FIGS. 4H and 4I, in terms of cellular experiments, after respectively transfecting inhibitors of miR-18a-3p, miR-497-5p, and miR-3074-5p into ATDC5 cells and primary human chondrocytes, it was found that only the miR-3074-5p inhibitor was capable of increasing the expression of aggrecan and type II collagen in both ATDC5 cells and primary human chondrocytes. In other words, in arthritic conditions, the microRNA miR-3074-5p serves as an essential mediator that controls the expression levels of aggrecan and type II collagen.

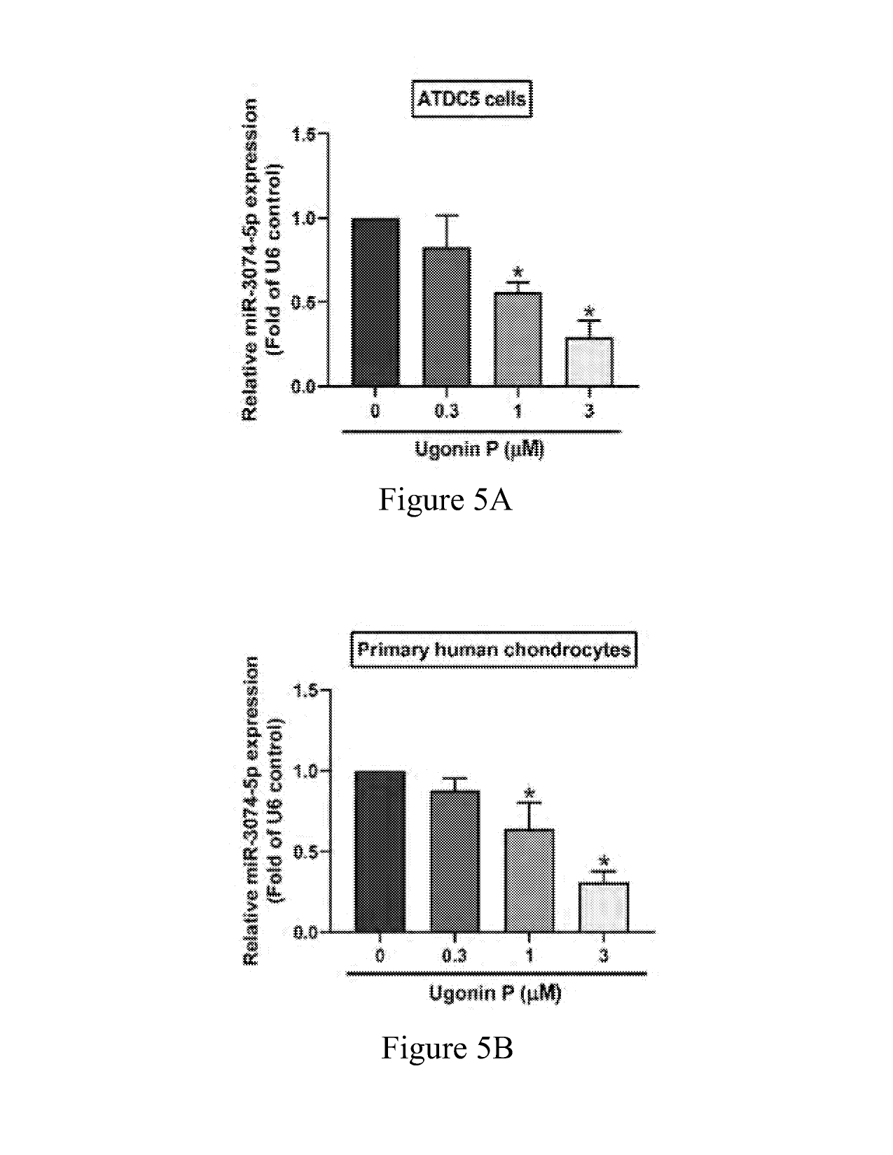

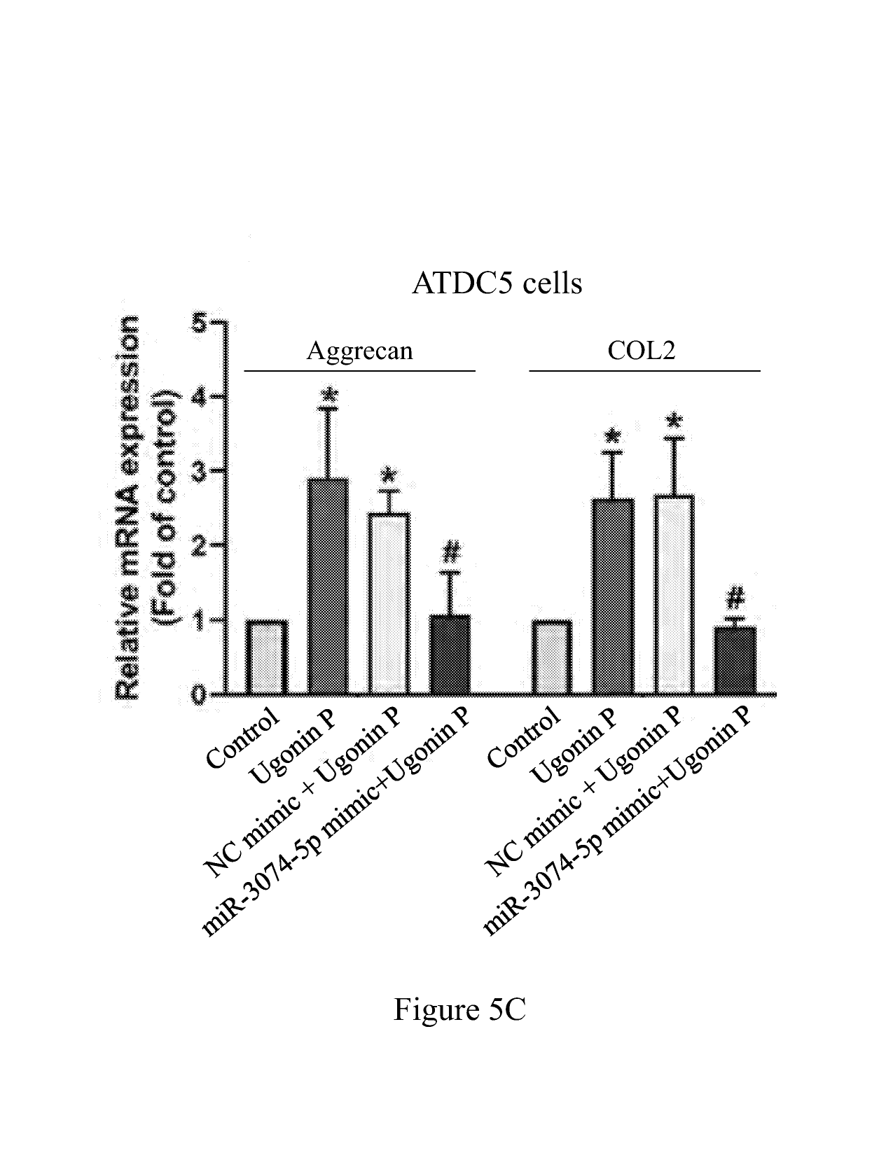

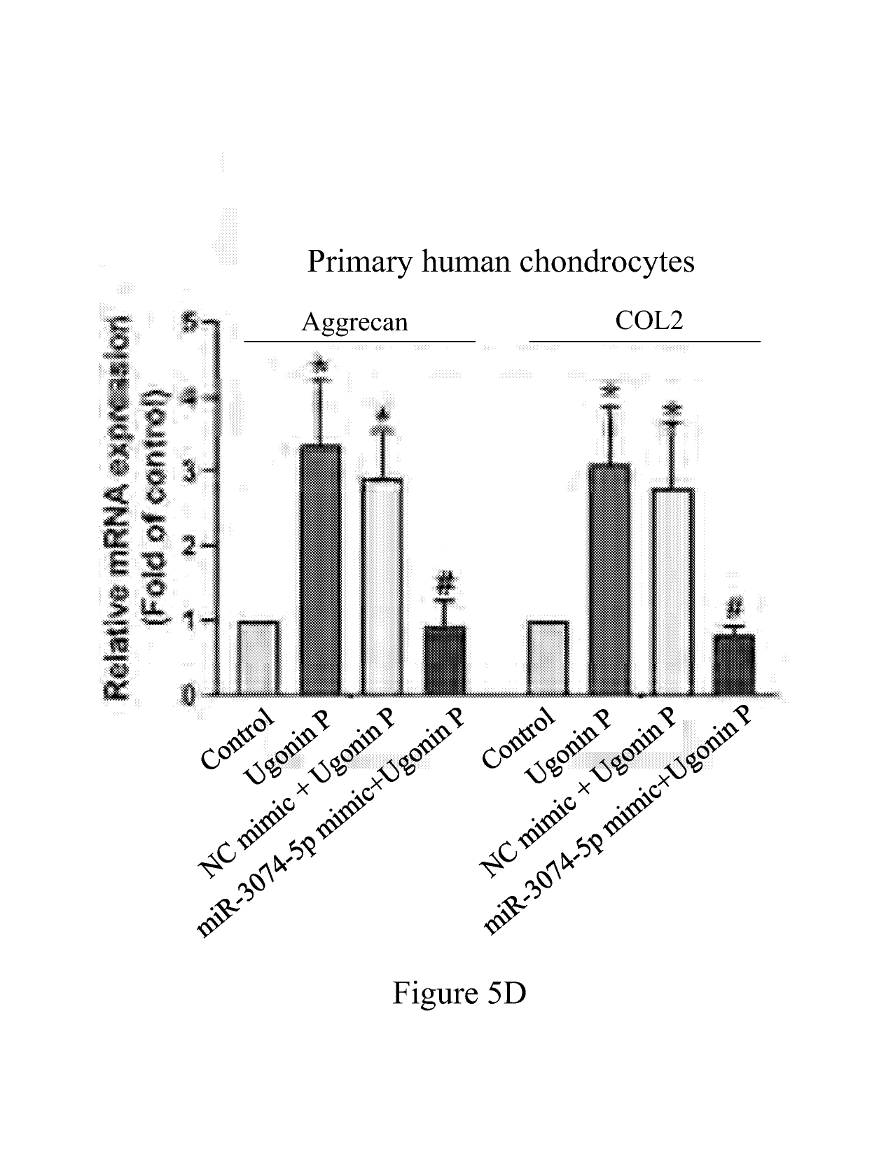

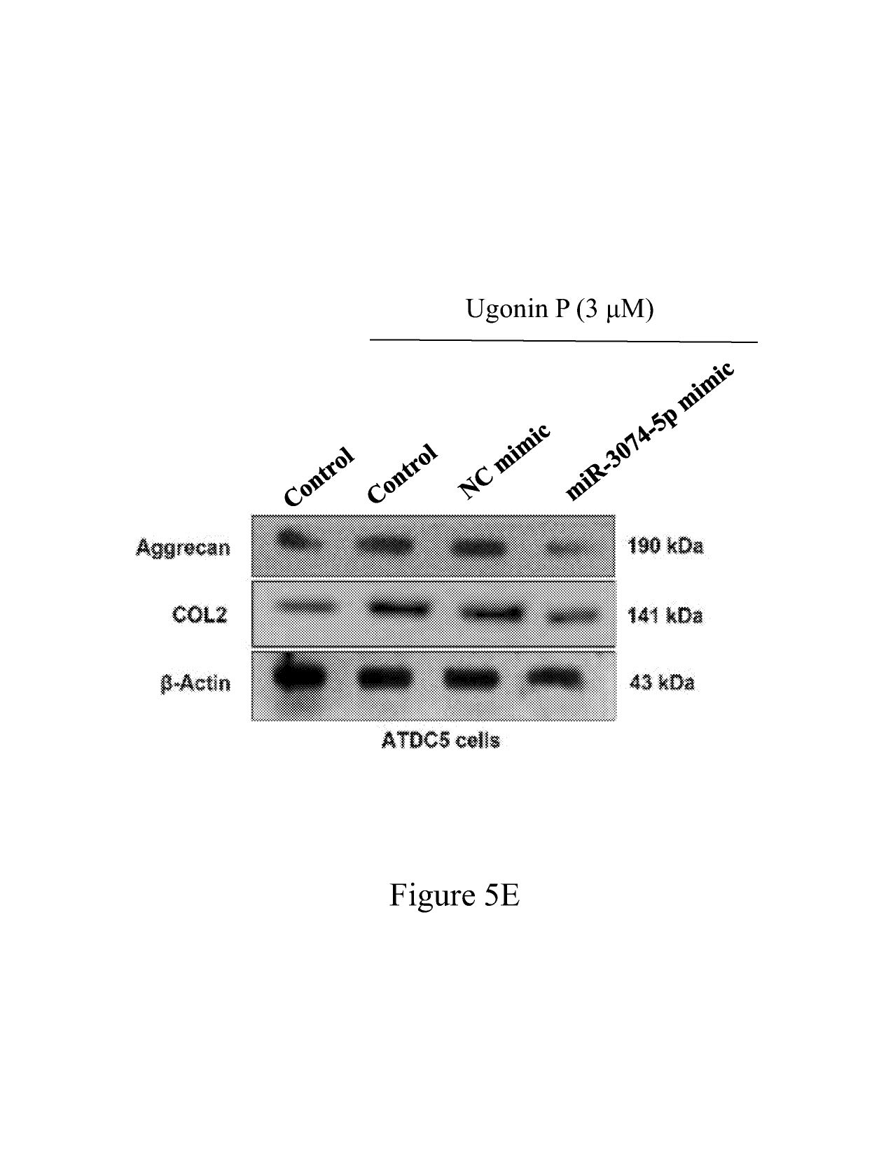

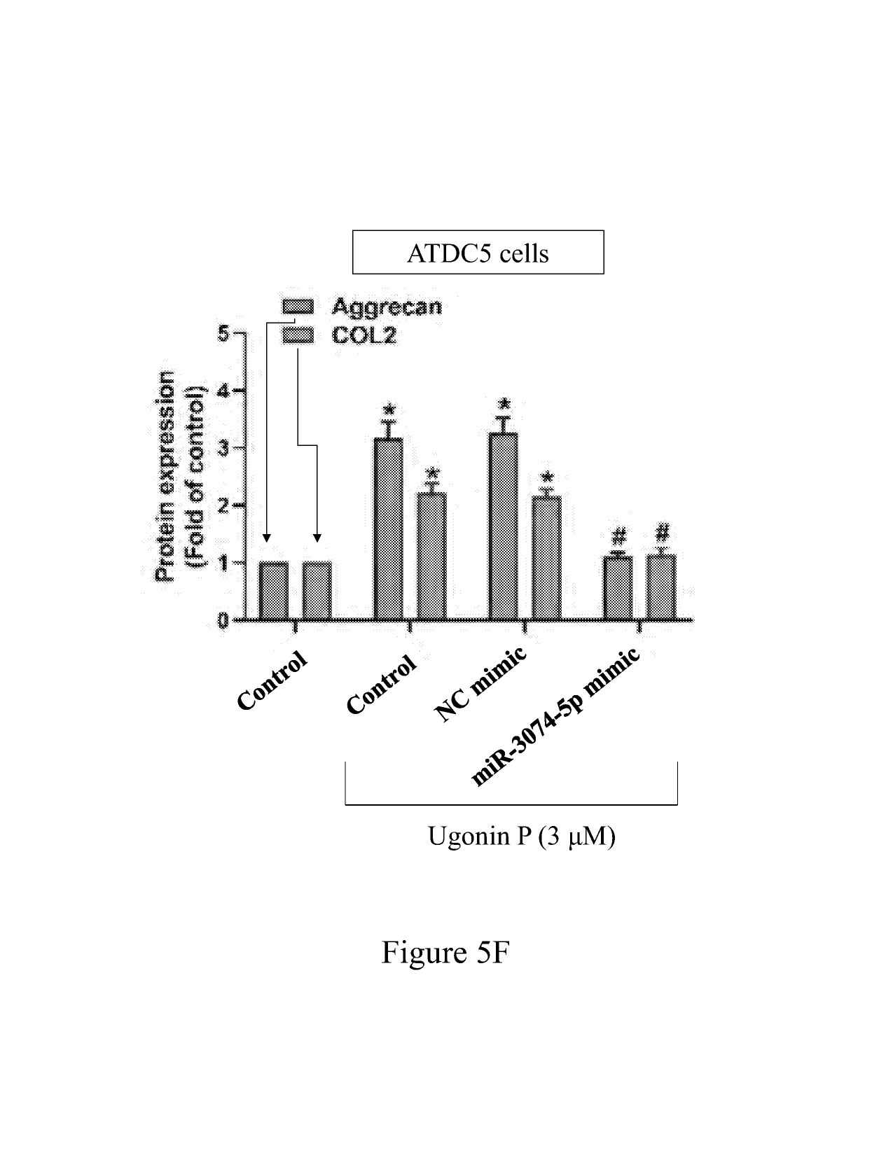

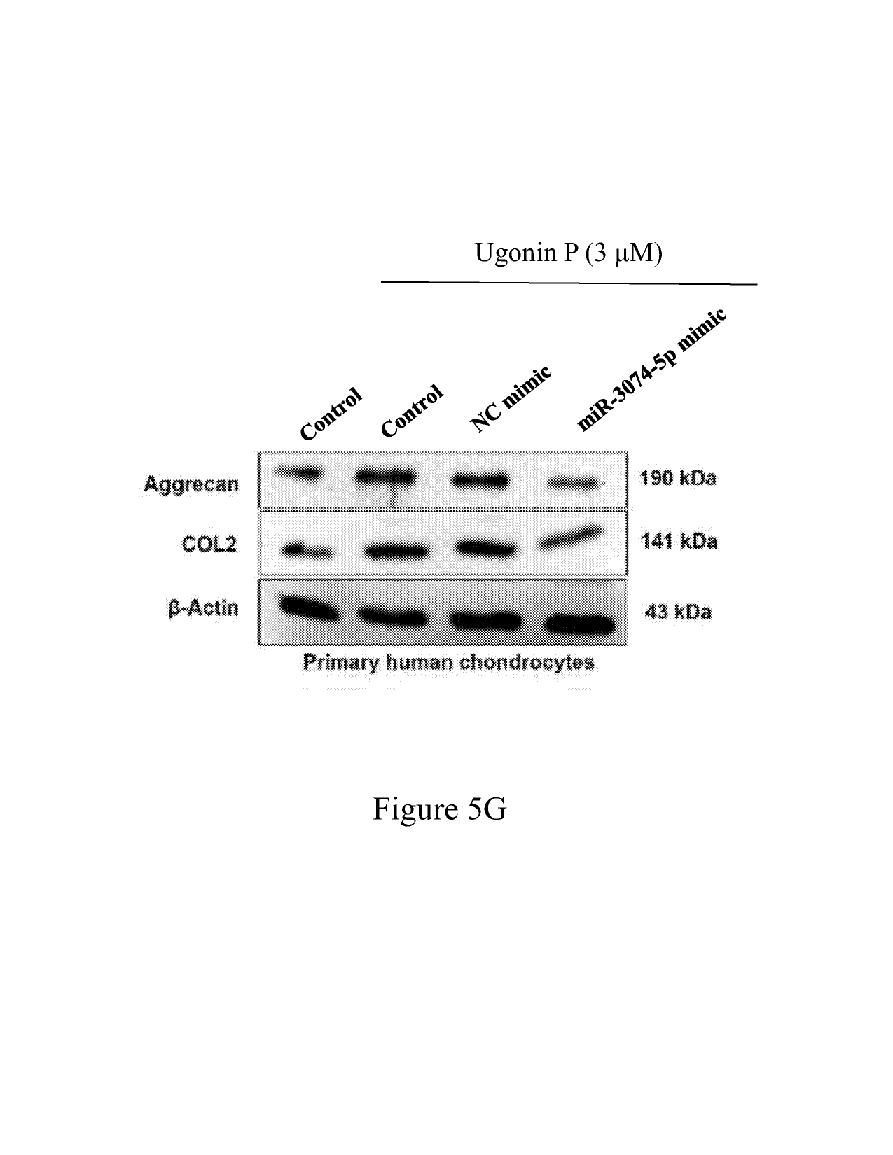

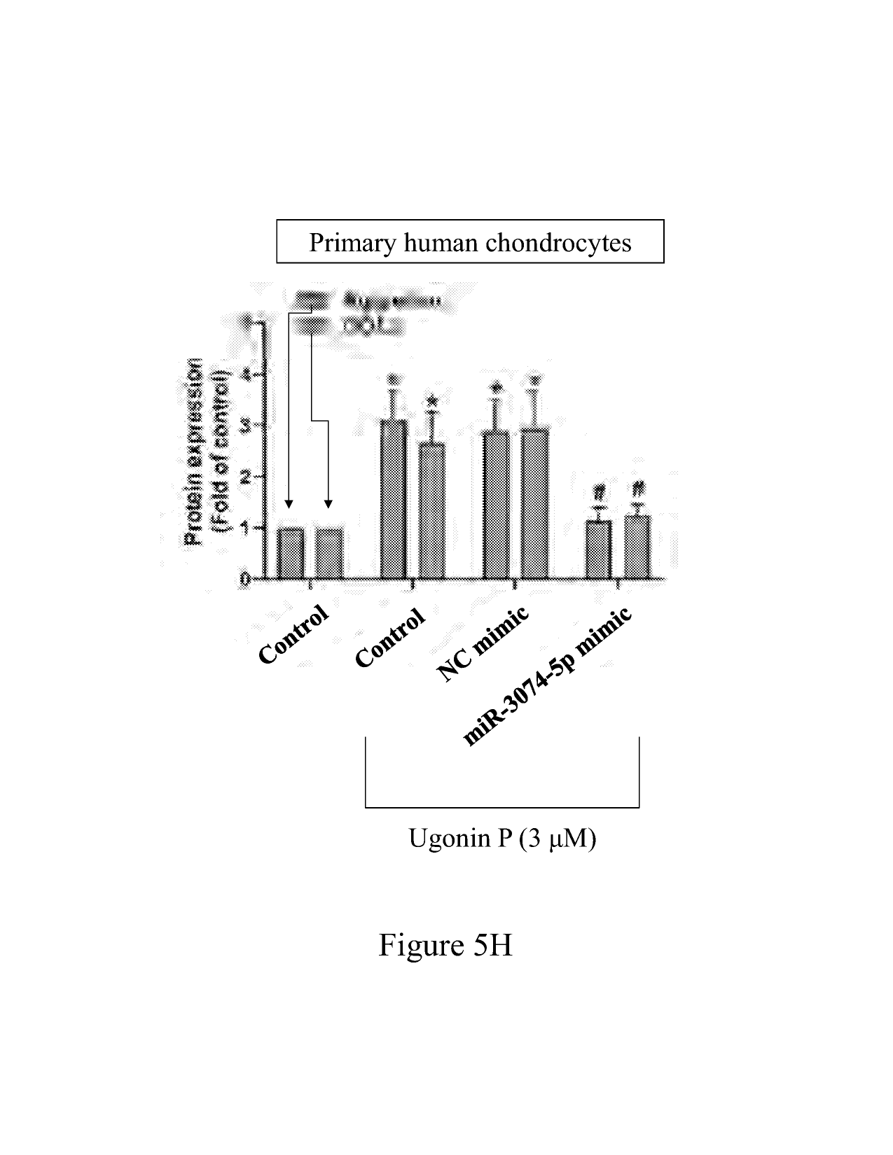

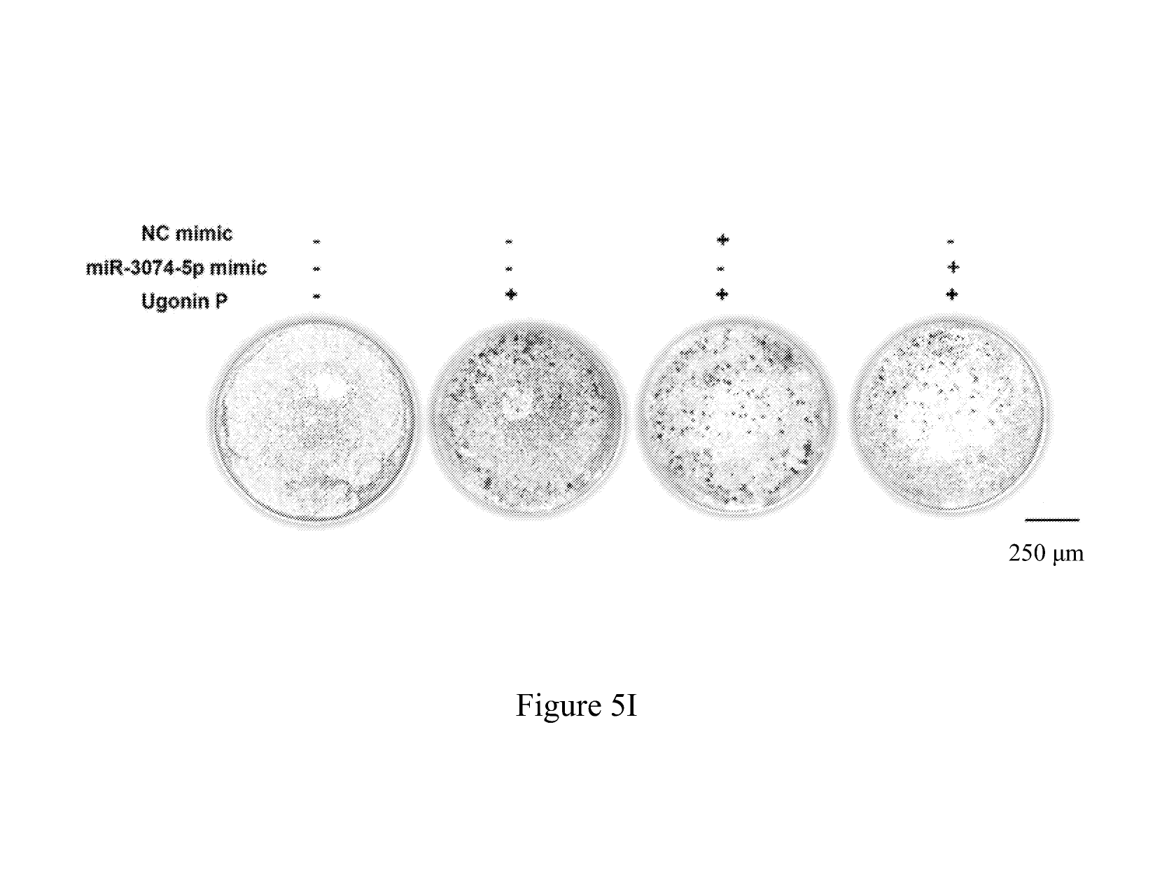

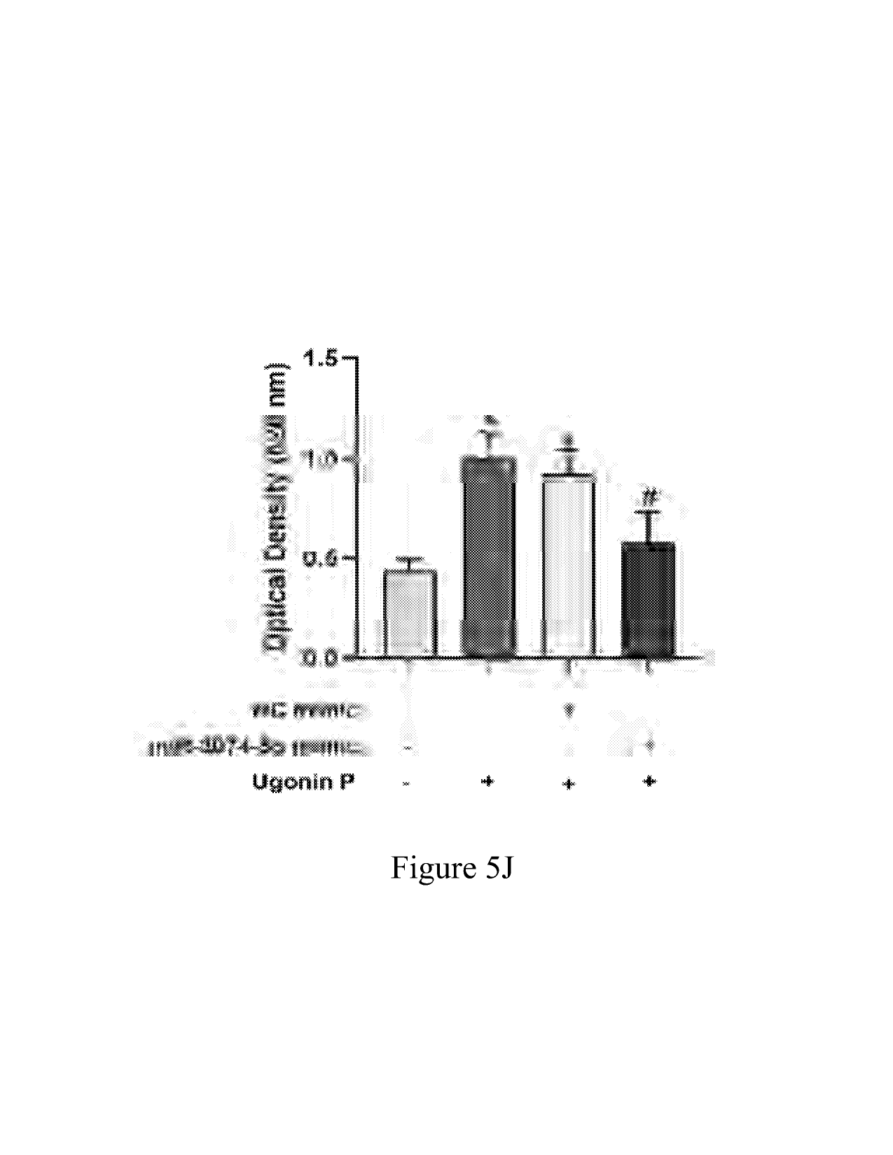

Next, this embodiment explores whether ugonin P can promote chondrogenesis by regulating miR-3074-5p. Referring to FIG. 5A and FIG. 5B, the study found that ugonin P indeed promotes chondrogenesis by inhibiting miR-3074-5p. Meanwhile, referring to FIGS. 5C to 5H, from another perspective, the administration of miR-3074-5p mimics (equivalent to increasing miR-3074-5p levels) can reverse the positive effects originally induced by Ugonin P, thereby decreasing the expression of aggrecan and type II collagen. Furthermore, as shown in FIG. 5I and FIG. 5J, the addition of miR-3074-5p mimics (50nM) also reduced the expected products of chondrogenesis. Therefore, it is understood that Ugonin P increases the expression of aggrecan and type II collagen by inhibiting the production of miR-3074-5p, thereby achieving chondrogenesis.

Example 4: ugonin P increases aggrecan and type II collagen via the MAPK signaling pathway.

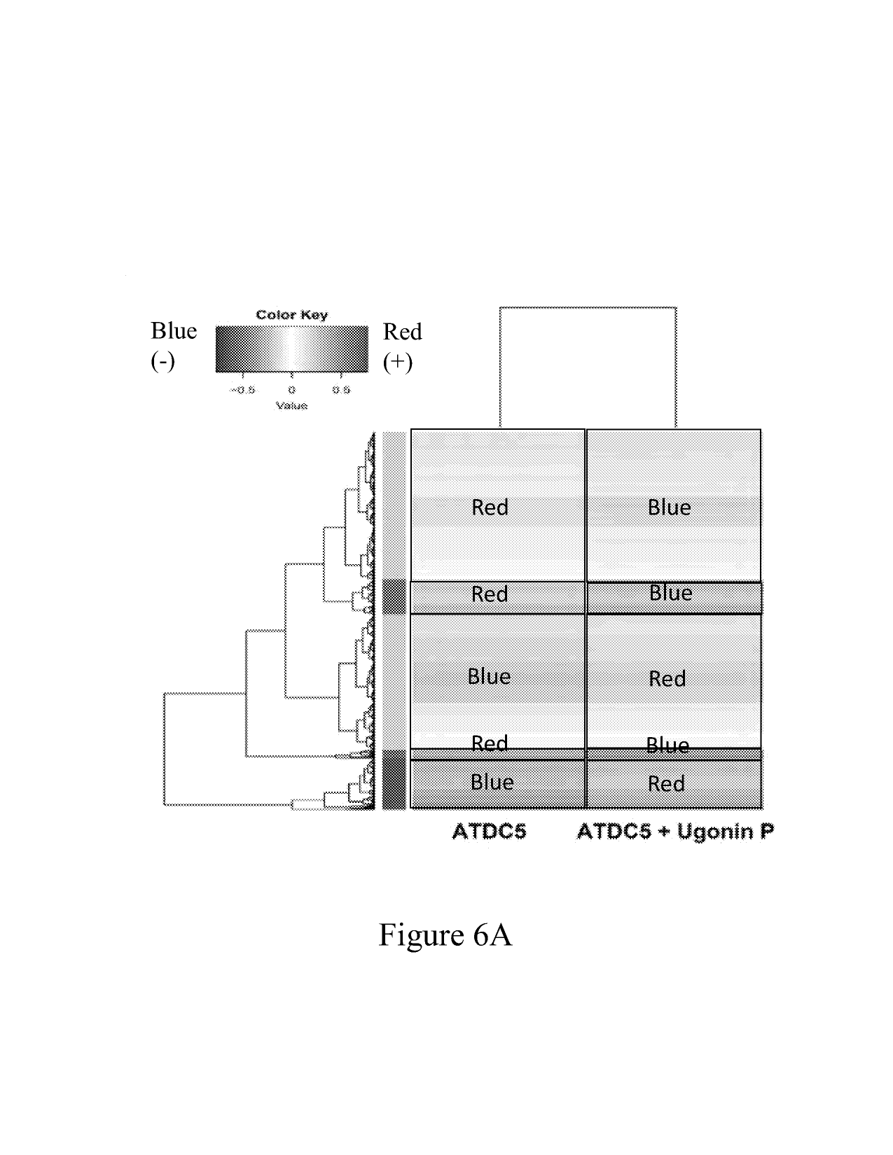

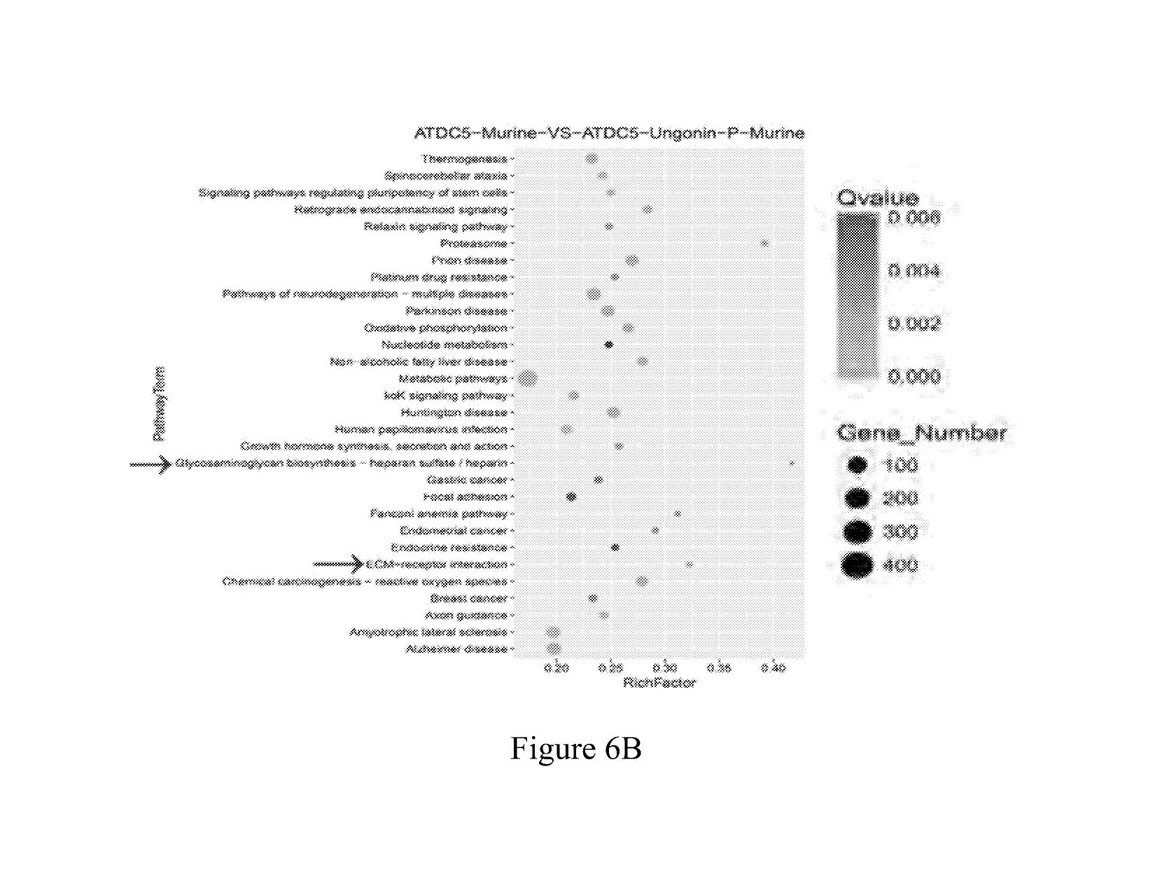

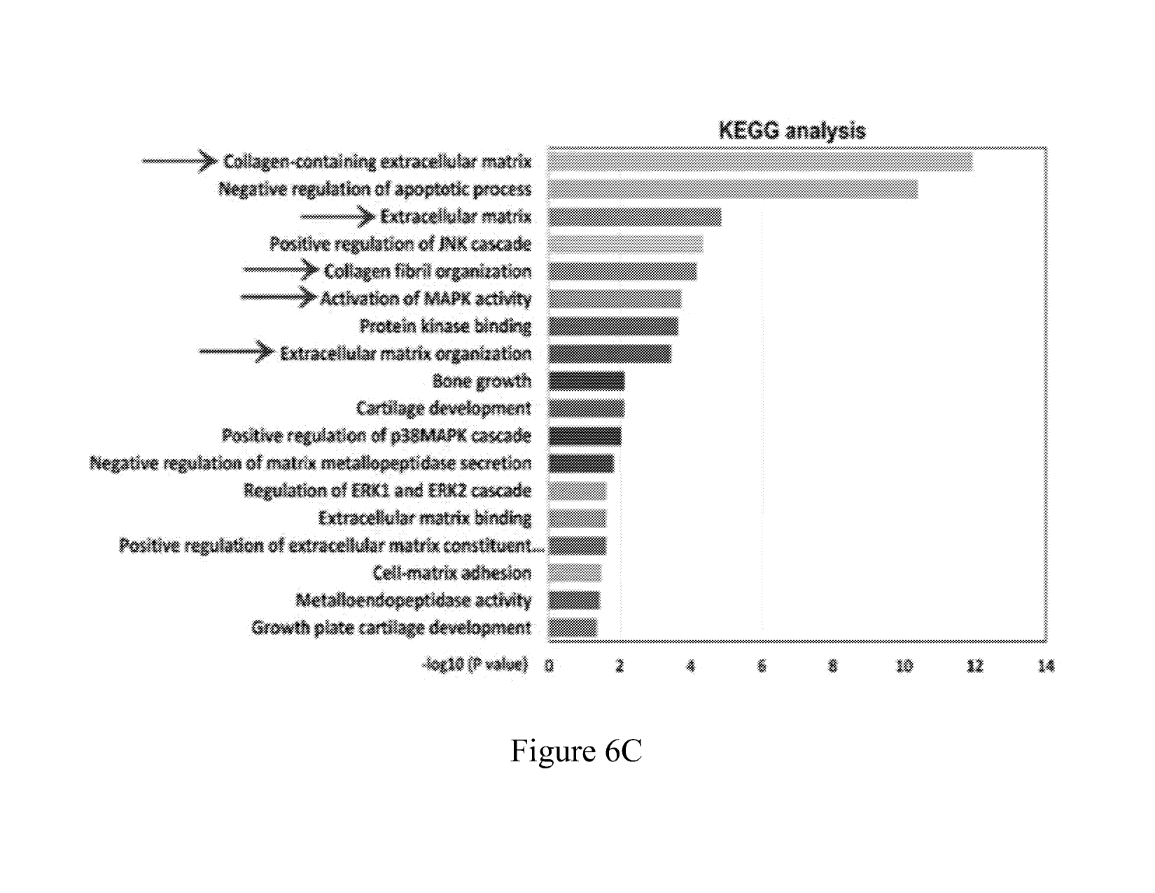

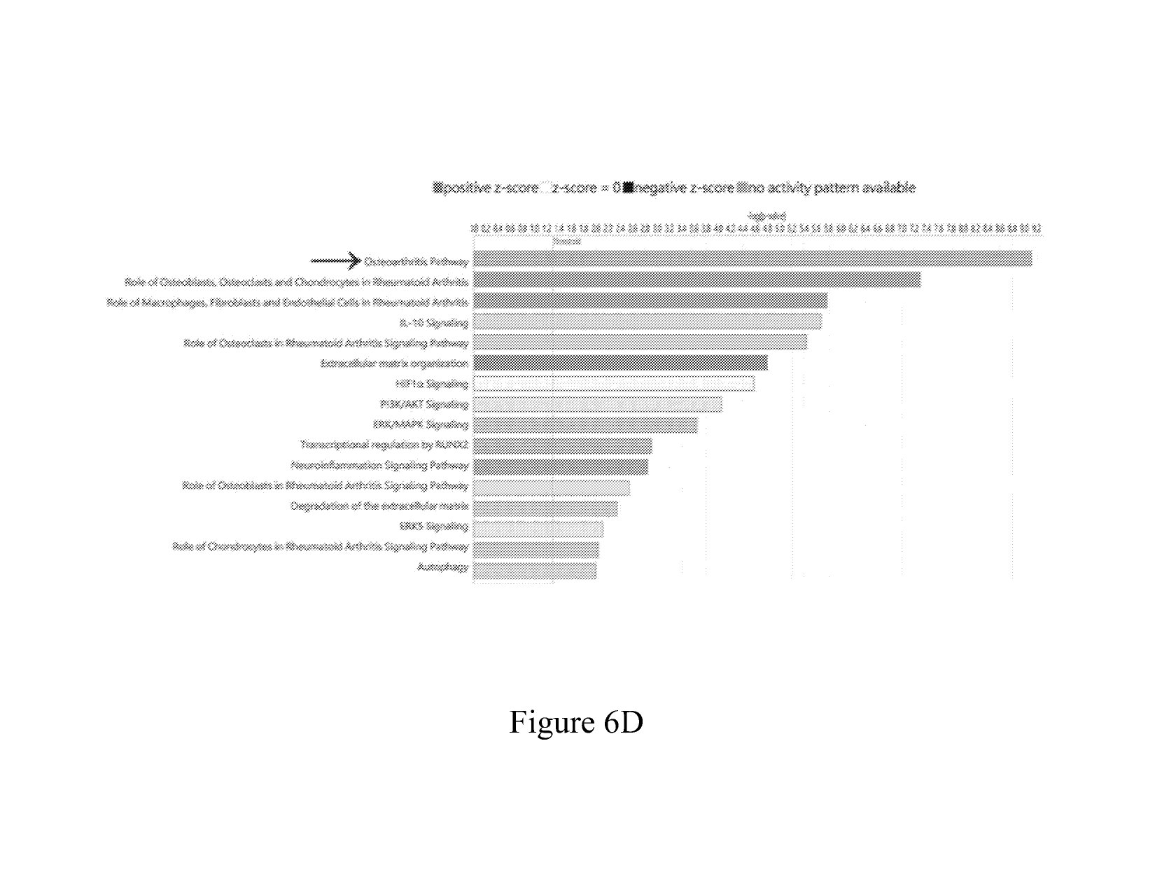

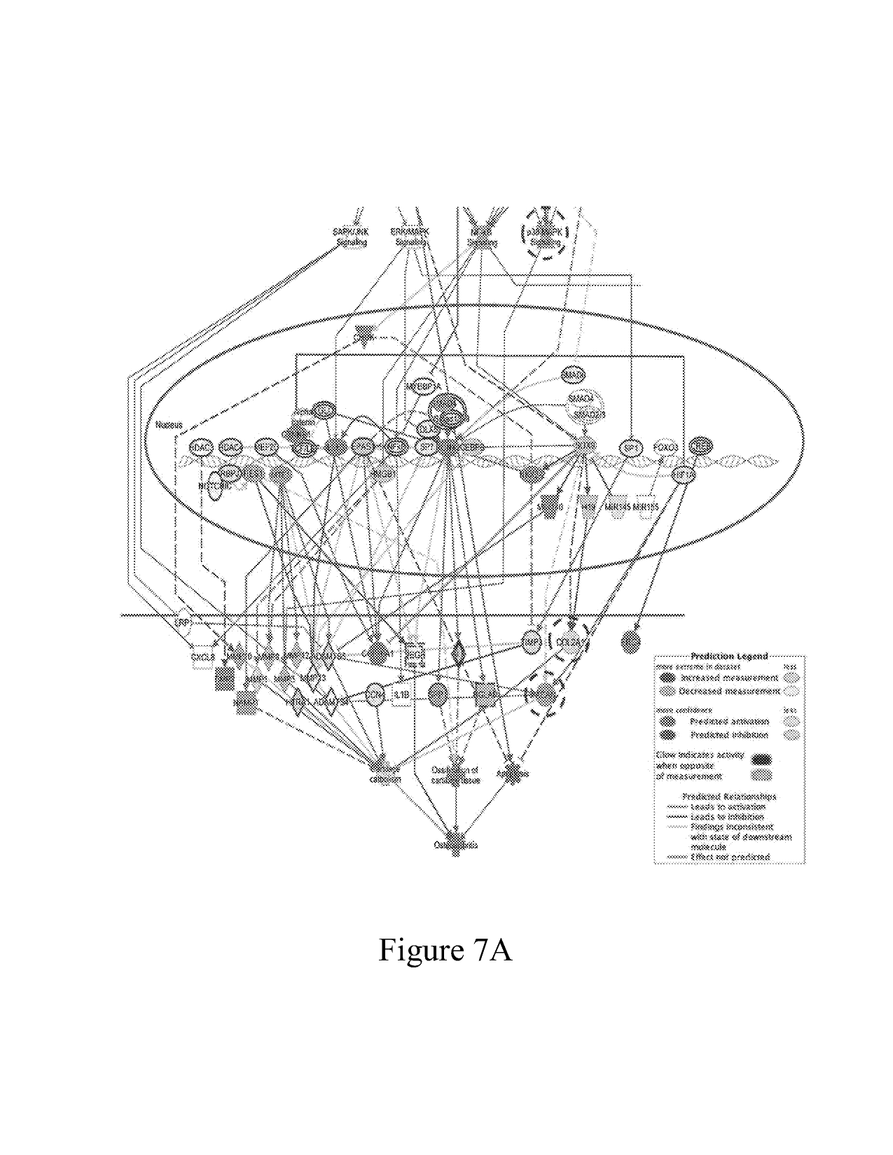

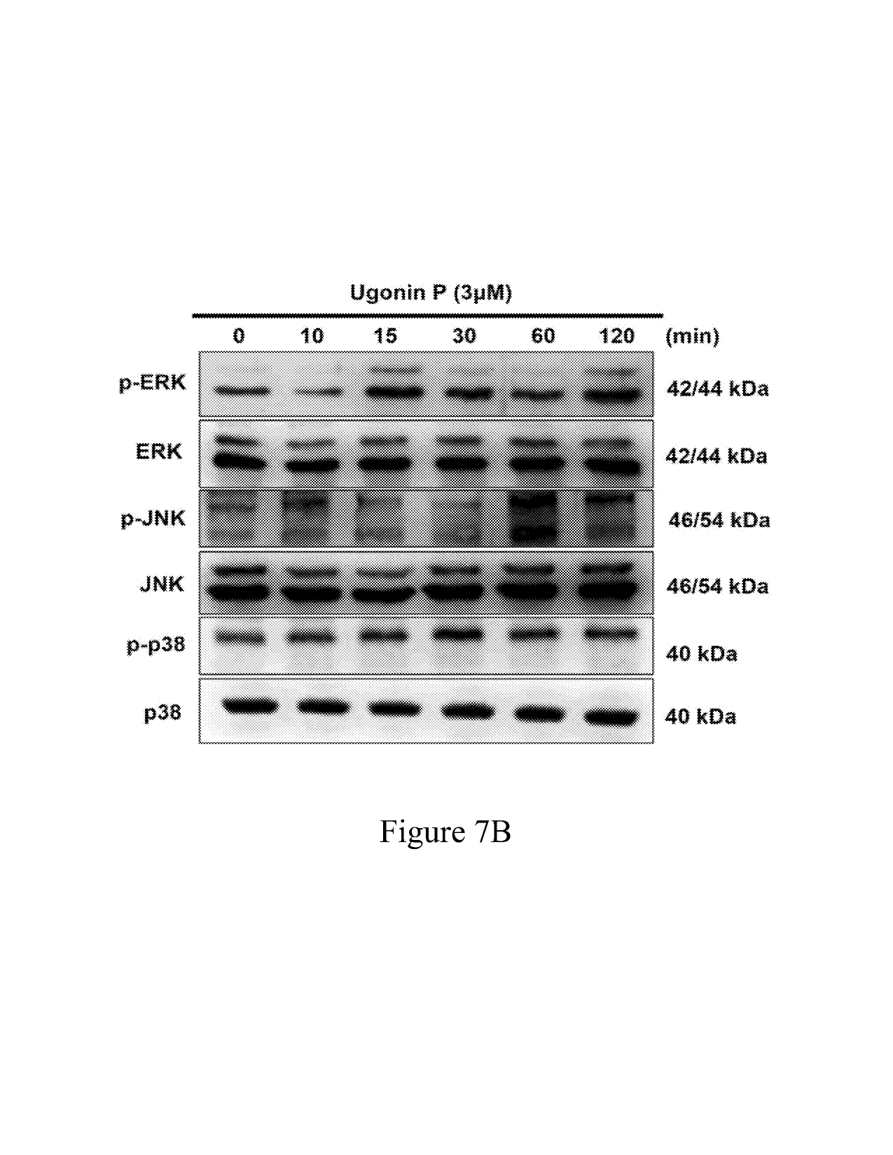

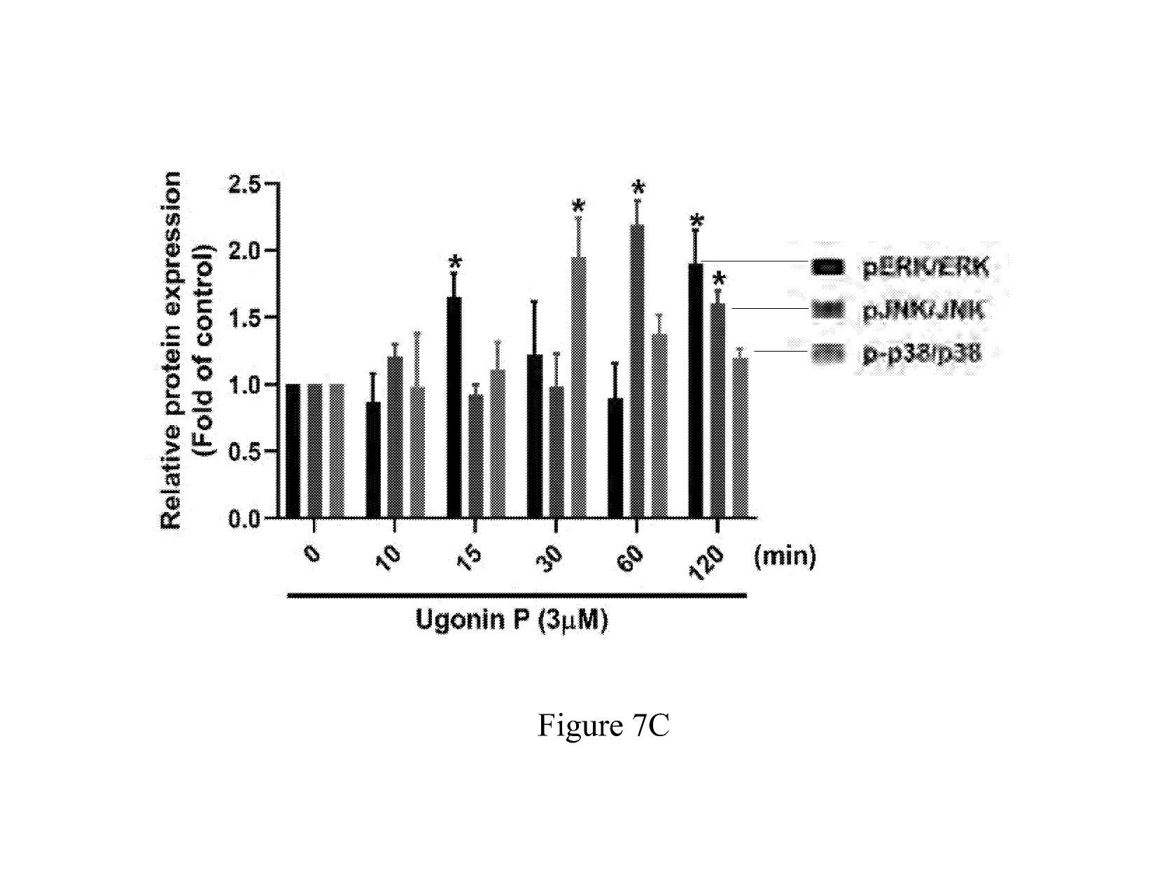

To explore the signaling pathways associated with ugonin-regulated chondrogenesis, one aspect of this embodiment involves treating ATDC5 cells with ugonin P and conducting RNA sequencing (RNA-seq) analysis, as illustrated in FIG. 6A. Subsequently, as shown in FIG. 6B and FIG. 6C, KEGG (Kyoto Encyclopedia of Genes and Genomes) analysis indicates that ugonin P enhances glycosaminoglycan biosynthesis, extracellular matrix (ECM) organization, and the MAPK signaling pathway. Furthermore, as shown in FIG. 6D and FIG. 7A, IPA (Ingenuity Pathway Analysis) reveals that among the osteoarthritis-related pathways, those specifically involving MAPK are more significantly upregulated.

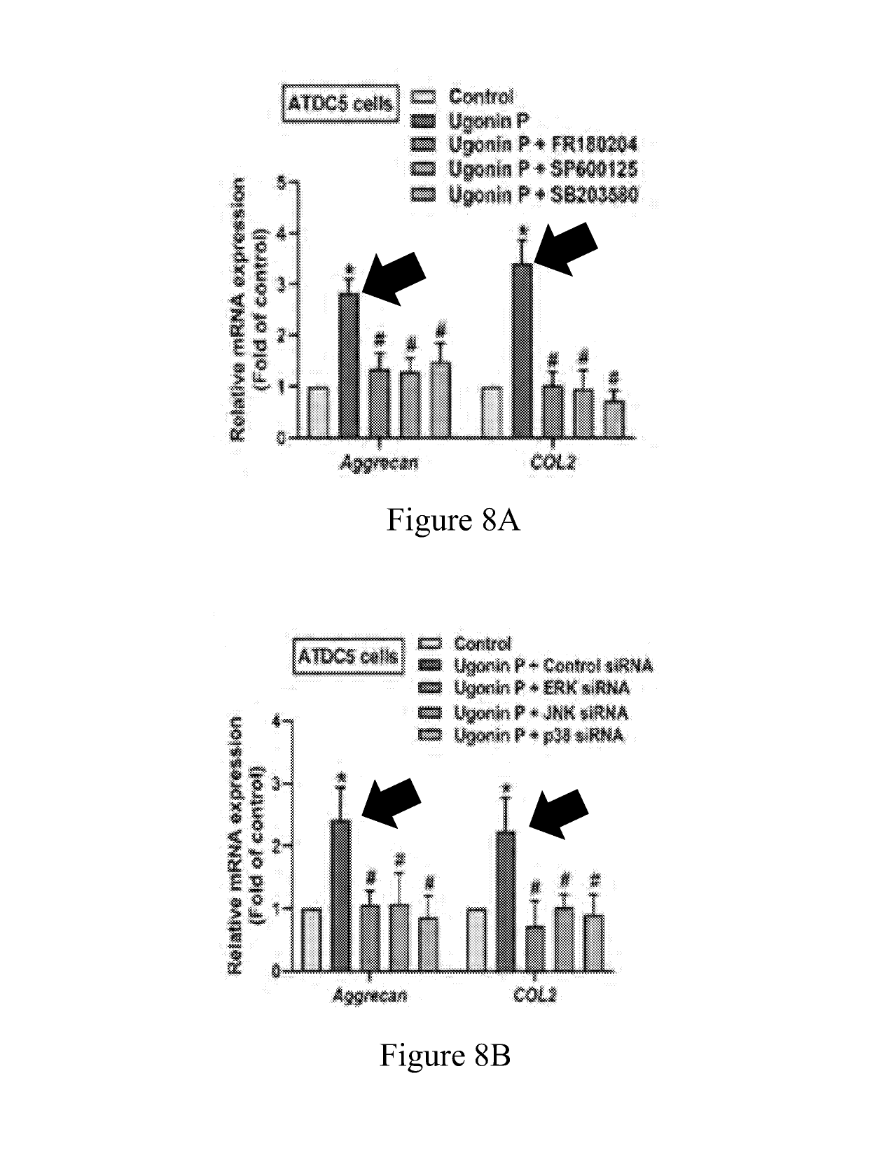

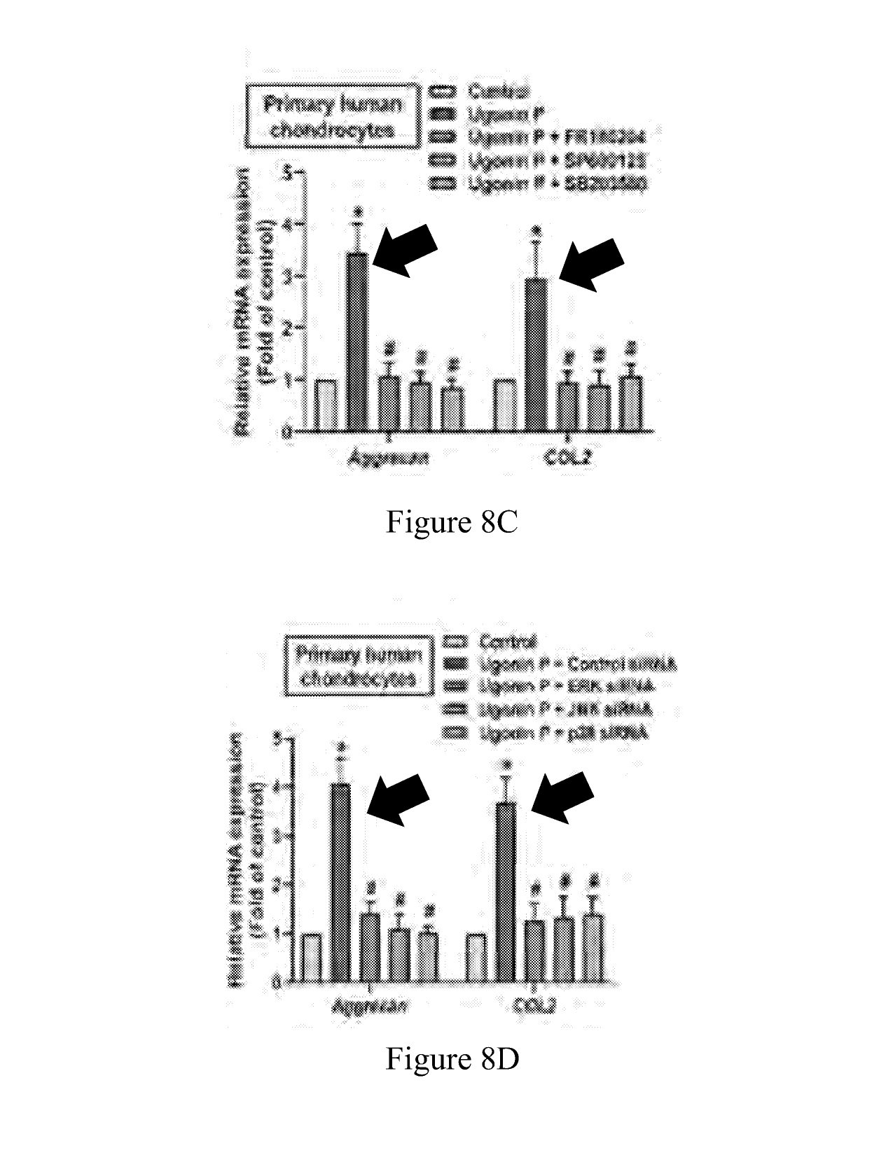

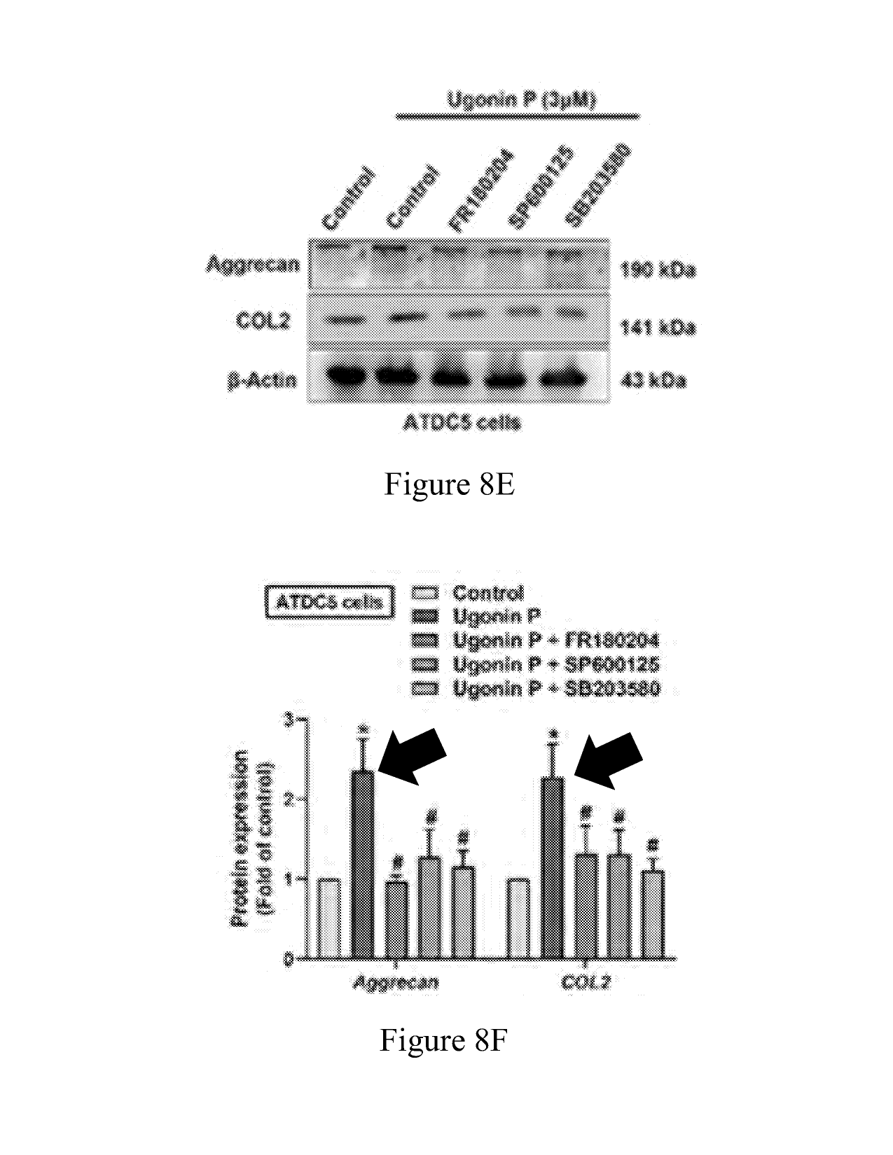

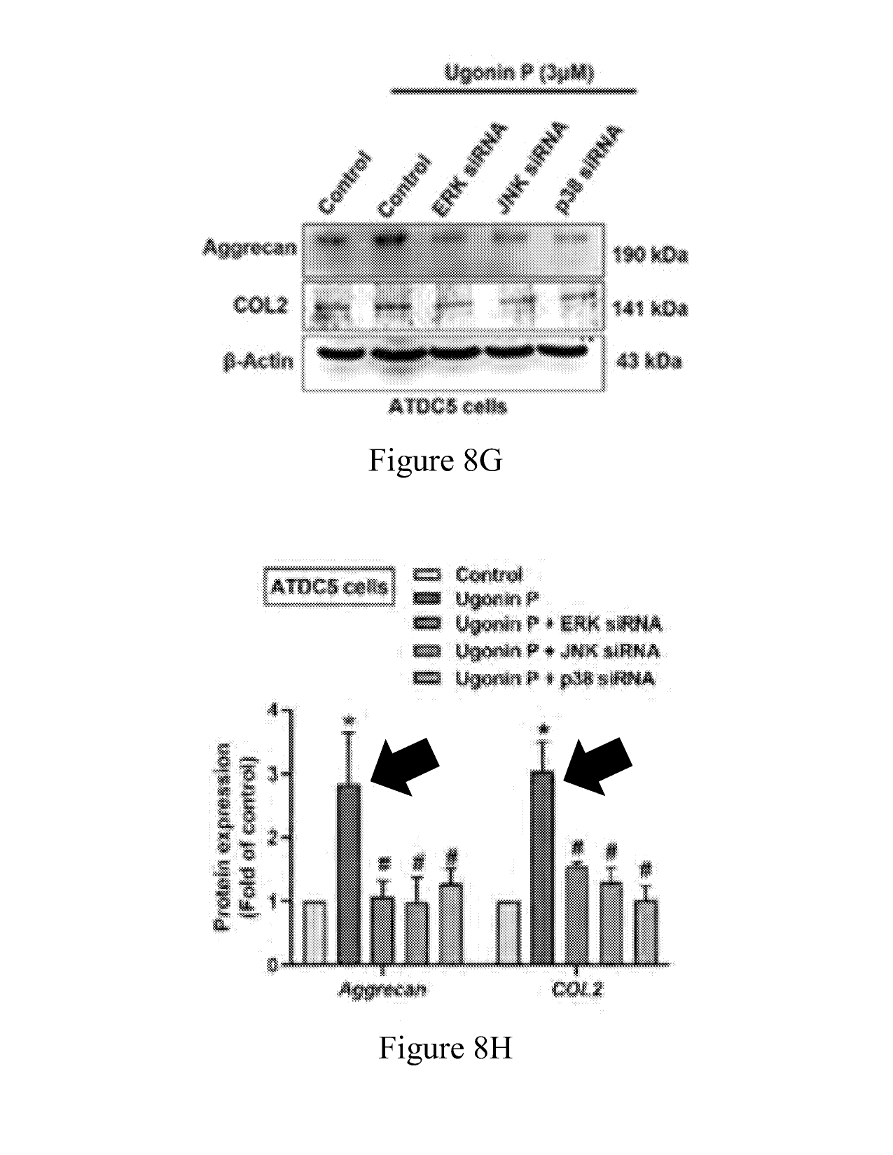

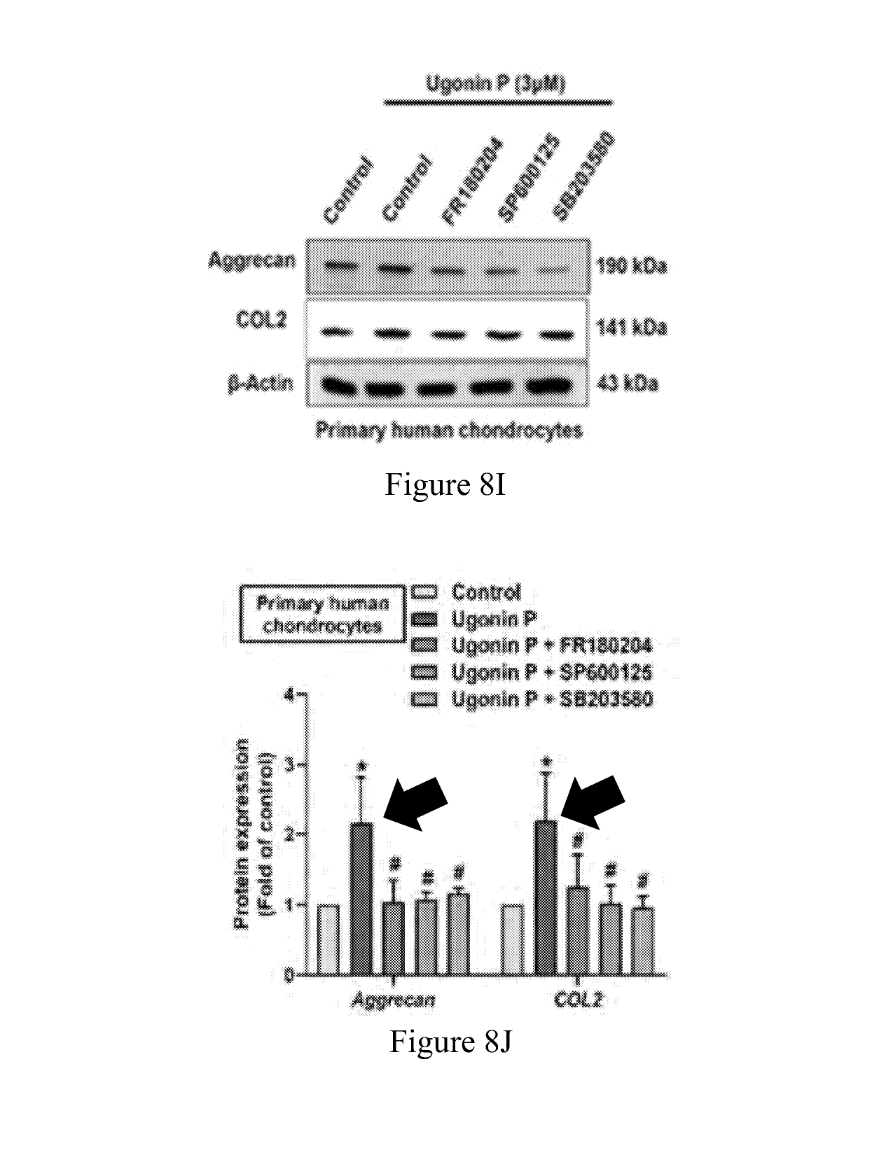

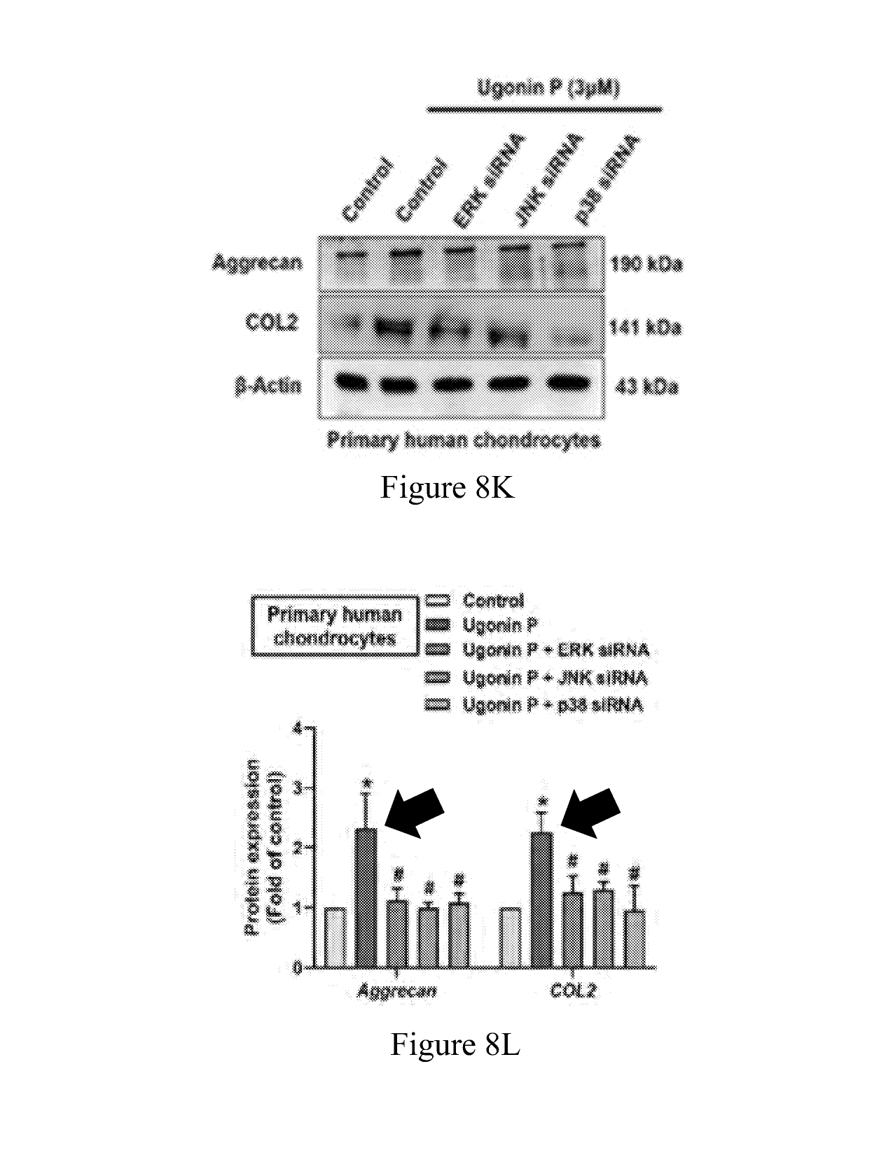

Furthermore, to validate these results, another aspect of this embodiment involves utilizing Western blot analysis to examine the role played by MAPK signaling in the stimulation induced by ugonin P. As shown in FIG. 7B and FIG. 7C, ugonin P induced the phosphorylation of ERK, JNK, and p38. Subsequently, referring to FIG. 8A to FIG. 8L, the use of inhibitors for ERK, JNK, and p38, or their respective siRNAs, reduced the expression of aggrecan and type II collagen induced by ugonin P. It can be observed that the groups indicated by arrows in FIG. 8A to FIG. 8L represent the ugonin P-treated groups, which in each figure are significantly higher than the control groups to their left (no ugonin P treatment) and the experimental groups to their right (treated with inhibitors or siRNA). In other words, in chondrocytes, the MAPK signaling pathway is involved in the process by which ugonin P induces the production of aggrecan and type II collagen.

In summary, the technical effects of the present invention include:

(1) Articular cartilage degradation frequently occurs in patients with osteoarthritis (OA) and rheumatoid arthritis (RA). Although various methods currently exist to treat the symptoms of these patients, none effectively slow down or arrest the disease progression. However, existing evidence suggests that improving the composition or properties of the extracellular matrix (ECM) can serve as a therapeutic approach. It is understood that the ECM is primarily composed of aggrecan and type II collagen, which are essential for maintaining the normal physiological functions of cartilage. The present invention first reveals, through GEO database analysis and clinical cases, that aggrecan and type II collagen are indeed significantly reduced in OA and RA patients; in other words, upregulating the expression of aggrecan and type II collagen in these patients represents a potential therapeutic strategy. Chondrogenesis is based on the differentiation of mesenchymal stem cells into chondrocytes, followed by the condensation of these chondrocytes. In this regard, the present invention utilizes ATDC5 cells—which are considered a superior in vitro model for studying chondrocyte differentiation due to their rapid and extensive growth, homogeneity, and potential to differentiate into chondrocytes—to perform differentiation and use Alcian Blue staining to demonstrate that Ugonin P effectively promotes chondrogenesis.

(2) On one hand, the present invention demonstrates, through the transfection of miR-3074-5p mimics, that the chondrogenesis-promoting effect of Ugonin P is attenuated, primarily due to the impact on aggrecan and type II collagen. On the other hand, bioinformatics analysis confirms that miR-3074-5p serves as a novel target for the treatment of OA and RA. This leads to the following conclusions: first, Ugonin P significantly downregulates the expression of miR-3074-5p, thereby enhancing its chondrogenesis-promoting ability; second, miR-3074-5p inhibitors are useful for the treatment of arthritis.

(3) Through RNA sequencing, the present invention reveals changes in gene expression induced by Ugonin P stimulation. Specifically, in ATDC5 cells, IPA analysis and inhibitor assays show that proteins involved in osteoarthritis-related mechanisms, including ERK, JNK, and p38, are all affected by Ugonin P stimulation, which in turn influences the promotion of aggrecan and type II collagen production.

(4) In summary, the present invention demonstrates that Ugonin P can be used to treat joint-related diseases via a mechanism that includes promoting chondrogenesis. More specifically, this enhancement of chondrogenesis results from the ability of Ugonin P to increase aggrecan and type II collagen expression. Furthermore, Ugonin P achieves these effects by inhibiting miR-3074-5p and activating the MAPK signaling pathway.

The aforementioned embodiments are merely exemplary of the present invention. It will be appreciated by those skilled in the art that various modifications, equivalents, and alterations may be made without departing from the technical concept of the present invention. Such modifications should likewise be regarded as falling within the scope of the present invention.

What is claimed is:

1. A method for treating a joint-related disease, comprising: administering to a subject in need a composition comprising a ugonin compound, wherein the ugonin compound enhances chondrogenesis.

2. The method as claimed in claim 1, wherein the ugonin compound is selected from the group consisting of ugonin J, ugonin K, ugonin L, ugonin M, ugonin N, ugonin O, ugonin P, ugonin S, ugonin T, ugonin U, and ugonin V.

3. The method as claimed in claim 2, wherein the ugonin compound is ugonin P.

4. The method as claimed in claim 1, wherein the joint-related disease is selected from the group consisting of arthritis, chondrodysplasia, chondral injury, chondromalacia patella, chondritis, and chondrolysis.

5. The method as claimed in claim 4, wherein the arthritis is selected from the group consisting of osteoarthritis, rheumatoid arthritis, metabolic arthritis, psoriatic arthritis, and ankylosing spondylitis.

6. The method as claimed in claim 1, wherein the composition promotes a cartilage-forming cell of the subject in need to generate an extracellular matrix, thereby enhancing chondrogenesis.

7. The method as claimed in claim 6, wherein the extracellular matrix comprises collagen, glycosaminoglycan, and proteoglycan.

8. The method as claimed in claim 7, wherein the collagen comprises type II collagen, and the proteoglycan comprises aggrecan.

9. The method as claimed in claim 6, wherein the composition suppresses expression of a microRNA in the cartilage-forming cell of the subject in need, thereby enhancing chondrogenesis.

10. The method as claimed in claim 9, wherein the microRNA comprises miR-3074-5p.

11. The method as claimed in claim 6, wherein the cartilage-forming cell comprises chondroblast, chondrocyte, or any combination thereof.

12. The method as claimed in claim 1, further comprising: administering the composition via an administration route to the subject in need to reach an effective concentration of the ugonin compound in a biofluid thereof.

13. The method as claimed in claim 12, wherein the administration route comprises intra-articular injection.

14. The method as claimed in claim 12, wherein the effective concentration ranges from 0.1 μM to 100 μM.

15. The method as claimed in claim 12, wherein the biofluid comprises synovial fluid.

16. A composition for treating a joint-related disease, the composition comprising a ugonin compound selected from the group consisting of ugonin J, ugonin K, ugonin L, ugonin M, ugonin N, ugonin O, ugonin P, ugonin S, ugonin T, ugonin U, and ugonin V.

17. The composition as claimed in claim 16, wherein the joint-related disease is selected from the group consisting of arthritis, chondrodysplasia, chondral injury, chondromalacia patella, chondritis, and chondrolysis.

18. The composition as claimed in claim 17, wherein the ugonin compound is ugonin P.

19. The composition as claimed in claim 16, wherein the ugonin compound enhances chondrogenesis.

20. The composition as claimed in claim 16, wherein the composition is in a dosage form formulated for intra-articular injection.
